(19) United States
(12) Patent Application Publication
(10) Pub. No.: US 2025/0001018 A1
(43) Pub. Date: Jan. 2, 2025

(54) MAGNETIC FERROFERRIC OXIDE NANOPARTICLE, AND PREPARATION METHOD THEREFOR AND USE THEREOF

(71) Applicants: SUZHOU ZHECI PHARMACEUTICAL TECHNOLOGY CO., LTD., Suzhou, Jiangsu (CN); SOUTH MEDICAL UNIVERSITY, Guangzhou, Guangdong (CN)

(72) Inventors: Zheyu SHEN, Suzhou, Jiangsu (CN); Xuanyi LU, Suzhou, Jiangsu (CN)

(73) Assignees: SUZHOU ZHECI PHARMACEUTICAL TECHNOLOGY CO., LTD., Suzhou, Jiangsu (CN); SOUTH MEDICAL UNIVERSITY, Guangzhou, Guangdong (CN)

(21) Appl. No.: 18/694,348

(22) PCT Filed: Sep. 16, 2022
(86) PCT No.: PCT/CN2022/119469

§ 371 Date: Mar. 21, 2024

(30) Foreign Application Priority Data
Jan. 21, 2022 (CN) 202210072699.7

(51) Int. Cl. A61K 49/18; A61K 49/12; A61K 49/14; B82Y 5/00; B82Y 40/00

(52) U.S. Cl. CPC A61K 49/18; A61K 49/12; A61K 49/14; B82Y 5/00; B82Y 40/00

(57) ABSTRACT

The present invention discloses a magnetic ferroferric oxide nanoparticle, and a preparation method therefor and the use thereof. The magnetic ferroferric oxide nanoparticle contains ferroferric oxide and a hydrophilic macromolecule, wherein the ferroferric oxide and the hydrophilic macromolecule are in at least one of the following relationships (1) and (2): (1) the hydrophilic macromolecule is adsorbed on the surface of the ferroferric oxide; and (2) the ferroferric oxide and the hydrophilic macromolecule are in the state of mutual embedding or occlusion. In the present invention, the hydrophilic macromolecule is used as a stabilizer, and ferrous ions and ferric ions form the magnetic ferroferric oxide nanoparticle by means of coprecipitation; and the magnetic ferroferric oxide nanoparticle has a relatively high longitudinal magnetic relaxation rate r₁, a relatively low transverse/longitudinal magnetic relaxation rate ratio (r₂/r₁), good water solubility, high stability, and good biocompatibility, and can be used as a contrast agent for T1-weighted magnetic resonance imaging (MRI) to improve the contrast and sensitivity of MRI.

### CROSS-REFERENCE TO RELATED APPLICATIONS

The present application is a national phase entry under 35 USC § 371 of International Application PCT/CN2022/119469, filed Sep. 16, 2022, which claims the benefit of and priority to Chinese Patent Application No. 202210072699.7, filed Jan. 21, 2022, the entire disclosures of which are incorporated herein by reference.

### TECHNICAL FIELD

The present disclosure relates to the technical field of biomedical materials, and particularly to a magnetic ferroferric oxide nanoparticle, and a preparation method therefor and use thereof.

### BACKGROUND

Magnetic resonance imaging (MRI) has a good imaging effect on soft tissues and may effectively distinguish lesions from healthy tissues, thus being widely used in clinical diagnosis. An MRI contrast agent can improve the contrast and sensitivity of MRI. At present, the contrast agent is used in more than 40% of MRI clinical diagnosis, and the MRI contrast agent has become an important part of magnetic resonance imaging. The MRI contrast agent may react with hydrogen proton, thus shortening longitudinal relaxation time (T₁) or transverse relaxation time (T₂) of the proton. The MRI contrast agent may be divided into two types according to imaging effects: T₁-weighted MRI contrast agent (positive contrast agent), which may generate a bright signal; T₂-weighted MRI contrast agent (negative contrast agent), which may generate a dark signal. Compared with the T₂ contrast agent, the T₁ contrast agent may generate the bright signal, which is more conducive to lesion diagnosis by clinicians, so that the T₁ contrast agent is mostly used in clinic.

An r₁ value and an r₂/r₁ ratio are two important parameters to characterize the imaging effect of the MRI contrast agent. The higher the r₁ value of the T₁ contrast agent is, the better the effect is, and the lower the r₂/r₁ ratio is, the better the effect is; and similarly, the higher the r₂ value of the T₂ contrast agent is, the better the effect is, and the higher the r₂/r₁ ratio is, the better the effect is. Generally, the T₁ contrast agent is a gadolinium-based contrast agent, and the T₂ contrast agent is an iron-based contrast agent. The T₁ contrast agent used in clinic is mainly a gadolinium-based chelate, which is a micromolecule drug, comprising Magnevist®, Gadavist®, Dotarem®, Omniscan®, Vasovist®, and the like. American Food and Drug Administration (FDA) warned that the gadolinium-based contrast agent for clinical use has brain deposition and nephrotoxicity, so that there are certain risks in clinical application.

Iron is one of essential elements for human body, and ferroferric oxide has excellent biocompatibility. Compared with the gadolinium-based chelate, the ferroferric oxide is safer and more reliable when used as the MRI contrast agent for clinical diagnosis. However, the ferroferric oxide mainly exists as the T₂ contrast agent, and its characteristic of generating the dark signal seriously affects the clinical application. An r₂ value of the ferroferric oxide is positively correlated with a particle size. With the increase of the particle size, the saturation magnetization (M_{z}) of the ferroferric oxide is also increased, and the r₂ value is also increased. However, the saturation magnetization of the ferroferric oxide (magnetic the ferroferric oxide nanoparticle (ES-MION)) with a particle size of nm or less is obviously reduced, and the r₂ value is lower, so that the ferroferric oxide may be used as the T₁ contrast agent. Therefore, the magnetic ferroferric oxide nanoparticle with higher r₁ value and lower r₂ value and the particle size less than 5 nm is expected to become a safe and reliable T₁ contrast agent with good imaging effect to be applied to clinical diagnosis. In the related art, the ferroferric oxide with the particle size of 5 nm or less is prepared from polymers such as polyvinyl alcohol (PVA), which shows the characteristics of the T₁ contrast agent, but the dispersibility is poor and the imaging effect is limited.

### SUMMARY

The present disclosure aims to solve at least one of the technical problems existing in the prior art. Therefore, the present disclosure provides a magnetic ferroferric oxide nanoparticle, which has a higher r₁ value and a lower r₂/r₁ ratio, good water solubility, high stability and good biocompatibility.

Meanwhile, the present disclosure further provides a preparation method for the magnetic ferroferric oxide nanoparticle and use of the magnetic ferroferric oxide nanoparticle.

Specifically, technical solutions used in the present disclosure are specifically as follows.

In a first aspect, the present disclosure provides a magnetic ferroferric oxide nanoparticle, wherein the magnetic ferroferric oxide nanoparticle comprises ferroferric oxide and a hydrophilic macromolecule, and the ferroferric oxide and the hydrophilic macromolecule are in at least one of the following relationships (1) and (2): (1) the hydrophilic macromolecule is adsorbed on a surface of the ferroferric oxide; and (2) the ferroferric oxide and the hydrophilic macromolecule are mutually embedded or occluded. The magnetic ferroferric oxide nanoparticle simultaneously has the following properties 1) to 4):
1) an average particle size is greater than 2 nm and less than 5 nm;
2) an electrokinetic potential is less than or equal to −10 mV;
3) a hydrodynamic diameter is less than or equal to 20 nm; and
4) a longitudinal magnetic relaxation rate r₁ value under a magnetic field intensity of 3.0 T is greater than 5 mM⁻¹ s⁻¹; and a longitudinal magnetic relaxation rate r₁ value under a magnetic field intensity of 1.0 T is greater than 10 mM⁻¹ s⁻¹.

The magnetic ferroferric oxide nanoparticle according to the present disclosure in the first aspect comprises at least the following beneficial effects.

In the present disclosure, the hydrophilic macromolecule is used as a stabilizer, and forms the magnetic ferroferric oxide nanoparticle (which may be synthesized by coprecipitation) with the ferroferric oxide by surface adsorption and mutual embedding or occlusion. The magnetic ferroferric oxide nanoparticle is a water-soluble nano-drug, and compared with the ferroferric oxides obtained by surface modification of existing ferroferric oxide with a macromolecule and the ferroferric oxides synthesized by an oil-phase method in the related art, the magnetic ferroferric oxide nanoparticle of the present disclosure has better water solubility and higher stability, can increase the r₁ value and reduce the r₂/r₁ ratio, and may be used as a safe and reliable T₁ contrast agent.

In some embodiments of the present disclosure, a Zeta potential of the magnetic ferroferric oxide nanoparticle is less than or equal to −30 mV; preferably, the Zeta potential is less than or equal to −35 mV; and more preferably, the Zeta potential is −60 mV to −35 mV.

In some embodiments of the present disclosure, a hydrodynamic diameter of the magnetic ferroferric oxide nanoparticle is less than or equal to 20 nm; and preferably, the hydrodynamic diameter is less than or equal to 15 nm.

In some embodiments of the present disclosure, the hydrophilic macromolecule comprises any one or a copolymer or mixture of several of a carboxylic acid-containing macromolecule, an amino-containing macromolecule, a hydroxyl-containing macromolecule, an amide-containing macromolecule and a polysaccharide.

In some embodiments of the present disclosure, a molecular weight of the hydrophilic macromolecules is 1,000 to 10,000, and a suitable macromolecule may be selected according to a specific condition.

In some embodiments of the present disclosure, the carboxylic acid-containing macromolecule comprises any one or more of polyglutamic acid, polyaspartic acid, polymaleic acid, poly(2-ethylacrylic acid) and polyepoxysuccinic acid.

In some embodiments of the present disclosure, the amino-containing macromolecule comprises any one or more of polylysine, polyhistidine, poly-L-arginine and polydimethyl diallyl ammonium chloride.

In some embodiments of the present disclosure, the hydroxyl-containing macromolecule comprises any one or more of polyserine, polythreonine, polytyrosine and tannic acid.

In some embodiments of the present disclosure, the amide-containing macromolecule comprises any one or more of polyglutamine, polyasparamide, polyacrylamide and polymethacrylamide.

In some embodiments of the present disclosure, the polysaccharide comprises one or two of hyaluronic acid and sodium alginate.

In a second aspect, the present disclosure provides a preparation method for the magnetic ferroferric oxide nanoparticle as described above, which comprises a step of:

coprecipitating ferrous ions and ferric ions to form the magnetic ferroferric oxide nanoparticle by using the hydrophilic macromolecule as a stabilizer.

After coprecipitation, the ferrous ions and the ferric ions react to form the ferroferric oxide, and the ferroferric oxide and the hydrophilic macromolecule form the magnetic ferroferric oxide nanoparticle by surface adsorption and mutual embedding or occlusion.

More specifically, the hydrophilic macromolecule is used as the stabilizer, so that the ferrous ions and the ferric ions are subjected to the coordination reaction with the hydrophilic macromolecule first, and then the alkali liquor is added for the coprecipitation reaction to obtain the magnetic ferroferric oxide nanoparticle.

In some embodiments of the present disclosure, the preparation method for the magnetic ferroferric oxide nanoparticle comprises the following steps of:

heating a hydrophilic macromolecule solution, then mixing the hydrophilic macromolecule solution after being subjected to heating with an iron ion mixed solution comprising ferrous ions and ferric ions for a coordination reaction, and then adding an alkali liquor for a coprecipitation reaction to obtain the magnetic ferroferric oxide nanoparticle.

In some embodiments of the present disclosure, in the iron ion mixed solution, a concentration of ferrous ions is 30 mM to 500 mM, preferably 50 mM to 250 mM, more preferably 150 mM to 250 mM, and further preferably 200 mM to 250 mM; and a concentration of ferric ions is 60 mM to 1,000 mM, preferably 100 mM to 500 mM, more preferably 300 mM to 500 mM, and further preferably 350 mM to 500 mM.

In some embodiments of the present disclosure, a concentration of the hydrophilic macromolecule solution is 0.1 mg/mL to 20 mg/mL, and preferably 0.5 mg/mL to 5 mg/mL.

In some embodiments of the present disclosure, a volume ratio of the hydrophilic macromolecule solution to the iron ion mixed solution is (1 to 500): 1, and preferably (10 to 50): 1.

In some embodiments of the present disclosure, the hydrophilic macromolecule solution and the iron ion mixed solution are both an aqueous solution.

In some embodiments of the present disclosure, the ferrous ions are obtained by hydrolysis of water-soluble ferrous salt, and the water-soluble ferrous salt comprises any one or more of ferrous chloride, ferrous nitrate, ferrous bromide and ferrous sulfate, and preferably the ferrous sulfate.

In some embodiments of the present disclosure, the ferric ions are obtained by hydrolysis of water-soluble ferric salt, and the water-soluble ferric salt comprises any one or more of ferric chloride, ferric nitrate, ferric bromide and ferric sulfate, and preferably the ferric chloride.

In some embodiments of the present disclosure, a pH value of the alkali liquor is 10 to 12, and preferably 11.5. After the alkali liquor is added into a mixed solution of the iron ion mixed solution and the hydrophilic macromolecule solution, a pH value of a reaction system is 8 to 10, and preferably 9.5.

In some embodiments of the present disclosure, the alkali liquor comprises at least one of sodium hydroxide and an aqueous solution thereof, potassium hydroxide and an aqueous solution thereof, and ammonia water, and preferably the ammonia water. Preferably, a mass concentration of the ammonia water is 0.5% to 5%, and preferably 1% to 4%.

In some embodiments of the present disclosure, the alkali liquor is in a form of solution, and a volume ratio of the alkali liquor to the hydrophilic macromolecule solution is 1:(2 to 10), and preferably 1:(4 to 6).

In some embodiments of the present disclosure, independently, the hydrophilic macromolecule solution is heated at a temperature of 25° C. to 100° C., preferably 100° C.; and the coprecipitation reaction is conducted at a temperature of 25° C. to 100° C., and preferably 100° C. In some embodiments of the present disclosure, the coprecipitation reaction lasts for more than 5 minutes, and preferably 40 minutes to 100 minutes.

In a third aspect, the present disclosure provides use of the magnetic ferroferric oxide nanoparticle as described above in preparing a magnetic resonance imaging contrast agent.

In some embodiments of the present disclosure, the MRI contrast agent is a longitudinal relaxation contrast agent (T₁ contrast agent).

Compared with the prior art, the present disclosure has the following beneficial effects.

The magnetic ferroferric oxide nanoparticle of the present disclosure has a higher r₁ value (>5 mM⁻¹ s⁻¹, 3.0 T; and >10 mM⁻¹ s⁻¹, 1.0 T), and a lower r₂/r₁ ratio (<13.0, 3.0 T), and may be compared with a gadolinium-based chelate contrast agent for clinical use. As an iron-based MRI contrast agent, the magnetic ferroferric oxide nanoparticle of the present disclosure is synthesized by the coprecipitation method with the hydrophilic macromolecule as the stabilizer, has a good imaging effect, a low signal-to-noise ratio, good water solubility, good stability and a small hydrodynamic diameter, and is beneficial for blood circulation. Moreover, the hydrophilic macromolecule used in the present disclosure is biodegradable, and has better biocompatibility and higher safety compared with the gadolinium-based chelate contrast agent for clinical use.

### BRIEF DESCRIPTION OF THE DRAWINGS

FIG. 1 A shows a transmission electron micrograph of γ-PGA-ES-MION6 in Example 1, and FIG. 1 B shows a particle size distribution diagram of γ-PGA-ES-MION6 in Example 1;

FIG. 2A shows a hydraulic diameter of γ-PGA-ES-MION6 in Example 1, and FIG. 2B shows a Zeta potential diagram of γ-PGA-ES-MION6 in Example 1;

FIG. 3A shows a longitudinal MRI relaxation rate of γ-PGA-ES-MION6 in Example 1, and FIG. 3B shows a transverse MRI relaxation rate of γ-PGA-ES-MION6 in Example 1;

FIG. 4 shows a saturation magnetization diagram of γ-PGA-ES-MION6 in Example 1;

FIG. 5A shows an X-ray photoelectron spectrogram of γ-PGA-ES-MION6 in Example 1, and FIG. 5B shows an X-ray diffraction diagram of γ-PGA-ES-MION6 in Example 1;

FIG. 6 shows an infrared absorption spectrogram of γ-PGA-ES-MION6 in Example 1;

FIG. 7A shows T₁-weighted MRI images of γ-PGA-ES-MION6 aqueous solutions at different concentrations in Example 1 and pure water, FIG. 7B shows corresponding MRI signal intensities of γ-PGA-ES-MION6 aqueous solutions at different concentrations in Example 1;

FIG. 8 is a MRI image showing a tumor-bearing mouse after being injected with γ-PGA-ES-MION6 in Example 1;

FIG. 9A shows a transmission electron micrograph of PASP-ES-MION9 in Example 2, and FIG. 9B shows a particle size distribution diagram of PASP-ES-MION9 in Example 2;

FIG. 10A shows a hydraulic diameter of PASP-ES-MION9 in Example 2; and FIG. 10B shows a Zeta potential diagram of PASP-ES-MION9 in Example 2;

FIG. 11A shows a longitudinal MRI relaxation rate of PASP-ES-MION9 in Example 2, and FIG. 11B shows a transverse MRI relaxation rate of PASP-ES-MION9 in Example 2;

FIG. 12A shows an X-ray photoelectron spectrogram of PASP-ES-MION9 in Example 2, and FIG. 12B shows an X-ray diffraction diagram of PASP-ES-MION9 in Example 2;

FIG. 13 shows an infrared absorption spectrogram of PASP-ES-MION9 in Example 2;

FIG. 14A shows T₁-weighted MRI images of PASP-ES-MION9 aqueous solutions at different concentrations in Example 2, and FIG. 14B shows corresponding MRI signal intensities of PASP-ES-MION9 aqueous solutions at different concentrations in Example 2;

FIG. 15 a MRI image showing a tumor-bearing mouse after being injected with PASP-ES-MION9 in Example 2;

FIG. 16A shows a longitudinal MRI relaxation rate of HPMA-ES-MION in Example 3, and FIG. 16B shows a transverse MRI relaxation rate of HPMA-ES-MION in Example 3;

FIG. 17A shows a longitudinal MRI relaxation rate of PEAA-ES-MION in Example 4, and FIG. 17B shows a transverse MRI relaxation rate of PEAA-ES-MION in Example 4;

FIG. 18A shows a longitudinal MRI relaxation rate of PESA-ES-MION in Example 5, and FIG. 18B shows a transverse MRI relaxation rate of PESA-ES-MION in Example 5;

FIG. 19A shows a longitudinal MRI relaxation rate of ε-PL-ES-MION in Example 6, and FIG. 19B shows a transverse MRI relaxation rate of ε-PL-ES-MION in Example 6;

FIG. 20 A shows a longitudinal MRI relaxation rate of PLH-ES-MION in Example 7, and FIG. 20B shows a transverse MRI relaxation rate of PLH-ES-MION in Example 7;

FIG. 21A shows a longitudinal MRI relaxation rate of PLR-ES-MION in Example 8, and FIG. 21B shows a transverse MRI relaxation rate of PLR-ES-MION in Example 8;

FIG. 22A shows a longitudinal MRI relaxation rate of PDDA-ES-MION in Example 9, and FIG. 22B shows a transverse MRI relaxation rate of PDDA-ES-MION in Example 9;

FIG. 23A shows a longitudinal MRI relaxation rate of PSer-ES-MION in Example 10, and FIG. 23B shows a transverse MRI relaxation rate of PSer-ES-MION in Example 10;

FIG. 24A shows a longitudinal MRI relaxation rate of PThr-ES-MION in Example 11, and FIG. 24B shows a transverse MRI relaxation rate of PThr-ES-MION in Example 11;

FIG. 25A shows a longitudinal MRI relaxation rate of PTyr-ES-MION in Example 12, and FIG. 25B shows a transverse MRI relaxation rate of PTyr-ES-MION in Example 12;

FIG. 26A shows a longitudinal MRI relaxation rate of TA-ES-MION in Example 13, and FIG. 26B shows a transverse MRI relaxation rate of TA-ES-MION in Example 13;

FIG. 27A shows a longitudinal MRI relaxation rate of PolyQ-ES-MION in Example 14, and FIG. 27B shows a transverse MRI relaxation rate of PolyQ-ES-MION in Example 14;

FIG. 28A shows a longitudinal MRI relaxation rate of PHEA-ES-MION in Example 15, and FIG. 28B shows a transverse MRI relaxation rate of PHEA-ES-MION in Example 15;

FIG. 29A shows a longitudinal MRI relaxation rate of PAM-ES-MION in Example 16, and FIG. 29B shows a transverse MRI relaxation rate of PAM-ES-MION in Example 16;

FIG. 30A shows a longitudinal MRI relaxation rate of PMAM-ES-MION in Example 17, and FIG. 30B shows a transverse MRI relaxation rate of PMAM-ES-MION in Example 17;

FIG. 31A shows a longitudinal MRI relaxation rate of HA-ES-MION in Example 18, and FIG. 31B shows a transverse MRI relaxation rate of HA-ES-MION in Example 18;

FIG. 32A shows a longitudinal MRI relaxation rate of SA-ES-MION in Example 19, and FIG. 32B shows a transverse MRI relaxation rate of SA-ES-MION in Example 19;

FIG. 33A shows a longitudinal MRI relaxation rate of γ-PGA/PASP-ES-MION in Example 20, and FIG. 33B shows a transverse MRI relaxation rate of γ-PGA/PASP-ES-MION in Example 20; and FIG. 34A shows a longitudinal MRI relaxation rate of HPMA/PASP-ES-MION in Example 21, and FIG. 34A shows a transverse MRI relaxation rate of HPMA/PASP-ES-MION in Example 21.

### DETAILED DESCRIPTION

Technical solutions of the present disclosure are further described in detail hereinafter with reference to specific examples. Raw materials used in the following examples may be commercially available conventionally, unless otherwise specified; and the processes are all conventional processes in the art, unless otherwise specified.

### Example 1

Preparation of Magnetic Ferroferric Oxide Nanoparticle (v-PGA-ES-MION):

20 mL of polyglutamic acid γ-PGA (Mw=2,000) aqueous solution was subjected to bubble deoxygenation with nitrogen in a three-necked flask for 1 hour, and then heated at 100° C. to reflux. 0.4 mL of a mixed aqueous solution containing FeSO₄ and FeCl₃ was added into the flask, and then added with 6 mL of ammonia water. The mixture reacted for 1 hour under magnetic stirring at 100° C., and was allowed to stand and cooled. The sample after the reaction was dialyzed and purified, and the final sample was marked as γ-PGA-ES-MION. Concentrations of the polyglutamic acid aqueous solution, the mixed aqueous solution and the ammonia water and corresponding samples were shown in Table 1.

Physical properties of the sample in Example 1 were characterized. A recovery rate of iron of the sample in Example 1 was calculated to be 96%, which indicated that a utilization rate of a raw material was high, so that a cost could be effectively reduced. The sample in Example 1 and commercially available products Gadavist and Magnevist were respectively prepared into 6 aqueous solutions with different concentrations, and in-vitro imaging tests were carried out by 1.0 T, clinical 3.0 T and 7.0 T MRI systems to obtain longitudinal relaxation time T₁ and transverse relaxation time T₂. A longitudinal magnetic relaxation rate r₁ and a transverse magnetic relaxation rate r₂ were calculated by the following formula (c is a concentration of a magnetic substance in a contrast agent, and T₁ is relaxation time, wherein i=1 or 2), and results were shown in Table 1.

$${r}_{i} = \frac{Δ ( 1 / {T}_{i} )}{Δ c}$$

TABLE 1
Synthesis of γ-PGA-ES-MION and MRI characterization results

| | | | | | Recovery | | | | |
|---|---|---|---|---|---|---|---|---|---|
| Name of | C_{γ-PGA} | C_{FeCl3} | C_{FeSO4} | C_{NH3•H2O} | rate of iron | H₀ | r₁ | r₂ | |
| sample | (mg/mL)^{a} | (mM)^{a} | (mM)^{a} | (%)^{a} | (%)^{b} | (T) | (mM⁻¹s⁻¹) | (mM⁻¹s⁻¹) | r₂/r₁ |
| γ-PGA-ES- | 2.0 | 500 | 250 | 28 | 98.0 | 3.0 | 8.59 | 268.9 | 31.3 |
| MION1 | | | | | | | | | |
| γ-PGA-ES- | 1.0 | 500 | 250 | 28 | 93.8 | 3.0 | 8.41 | 285.9 | 34.0 |
| MION2 | | | | | | | | | |
| γ-PGA-ES- | 0.5 | 500 | 250 | 28 | 78.0 | 3.0 | 7.04 | 221.7 | 31.5 |
| MION3 | | | | | | | | | |
| γ-PGA-ES- | 0.25 | 500 | 250 | 28 | 25.4 | 3.0 | 7.26 | 247.9 | 34.2 |
| MION4 | | | | | | | | | |
| γ-PGA-ES- | 2.0 | 500 | 250 | 0.5 | 92.1 | 3.0 | 2.79 ± 0.06 | 47.5 ± 0.2 | 17.0 ± 0.3 |
| MION5 | | | | | | | | | |
| γ-PGA-ES- | 2.0 | 500 | 250 | 1 | 96.1 | 3.0 | 5.70 ± 0.04 | 70.2 ± 1.5 | 12.3 ± 0.2 |
| MION6 | | | | | | 1.0 | 10.11 ± 0.69 | 103.6 ± 4.5 | 10.3 ± 1.1 |
| γ-PGA-ES- | 2.0 | 500 | 250 | 2 | 95.3 | 3.0 | 6.42 ± 0.03 | 104.3 ± 0.4 | 16.3 ± 0.1 |
| MION7 | | | | | | | | | |
| γ-PGA-ES- | 2.0 | 500 | 250 | 4 | 98.7 | 3.0 | 6.94 ± 0.04 | 119.4 ± 2.8 | 17.2 ± 0.4 |
| MION8 | | | | | | | | | |
| Gadavist | — | — | — | | — | 3.0 | 4.64 | 4.81 | 1.04 |
| Magnevist | — | — | — | | — | 3.0 | 4.58 | 4.98 | 1.09 |

^{a}is a concentration of a reactant added before the reaction; and
^{b}is a molar percentage of an iron content of the ferroferric oxide in the raw material iron added.

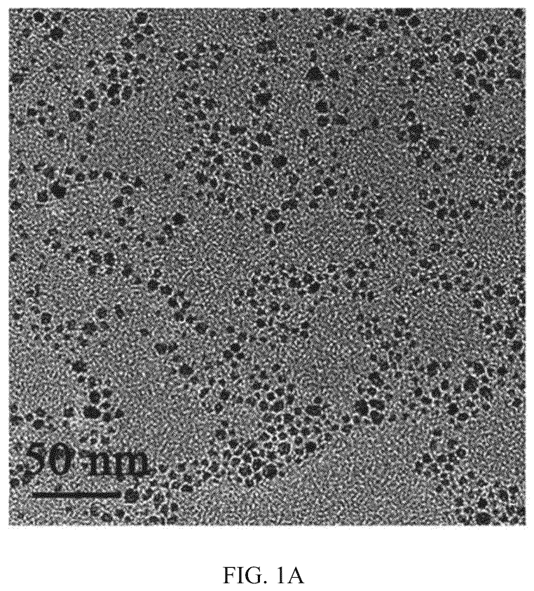

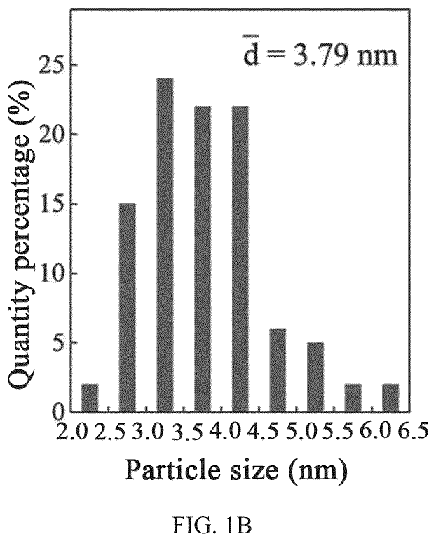

Specifically, FIG. 1A shows a transmission electron micrograph (TEM) of the sample γ-PGA-ES-MION6 in Example 1, and FIG. 1B shows a particle size distribution diagram of the sample γ-PGA-ES-MION6 in Example 1. It can be seen from the transmission electron micrograph of FIG. 1A that the nanoparticles are uniform in size and evenly dispersed, and particle size distributions of 100 nanoparticles randomly selected from FIG. 1A are counted to obtain the distribution diagram of FIG. 1B. It can be seen from the distribution diagram that the particle size of the sample is mainly about 3.5 nm. An average particle size of the sample γ-PGA-ES-MION6 is less than 5 nm, and the sample has the potential to be the T₁ contrast agent from the view of particle size. FIG. 2A shows a hydraulic diameter of the sample γ-PGA-ES-MION6 in Example 1, and FIG. 2B shows a Zeta potential diagram of the sample γ-PGA-ES-MION6 in Example 1. A hydraulic diameter of the sample is 8.7 nm, and a polymer dispersity index (PDI) of the sample is 0.265, which indicates good dispersity. A Zeta potential of the sample is −37.3 mV, and water phase dispersity of the sample can be improved by charge repulsion, which is beneficial for blood circulation of the sample.

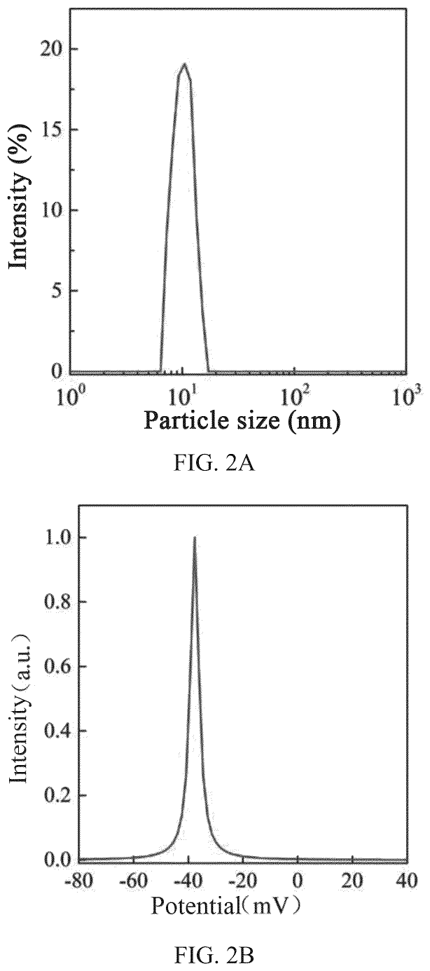

The concentrations of the polyglutamic acid aqueous solution, the mixed aqueous solution and the ammonia water have a great influence on the MRI performance of the sample. Under different synthetic conditions, the sample γ-PGA-ES-MION6 has the best comprehensive MRI performance. FIG. 3A and FIG. 3B show MRI relaxation rates of the sample γ-PGA-ES-MION6 in Example 1 (three samples γ-PGA-ES-MION6-1, γ-PGA-ES-MION6-2 and γ-PGA-ES-MION6-3 were tested in parallel for three times). A slope of a fitted line is a corresponding magnetic relaxation rate. It can be seen from FIG. 3A or FIG. 3B that goodness of linear fitting is 0.999 or more, which proves that the sample has an excellent linear relationship, thus indicating that the relaxation time may be changed linearly with a change of a concentration of the sample.

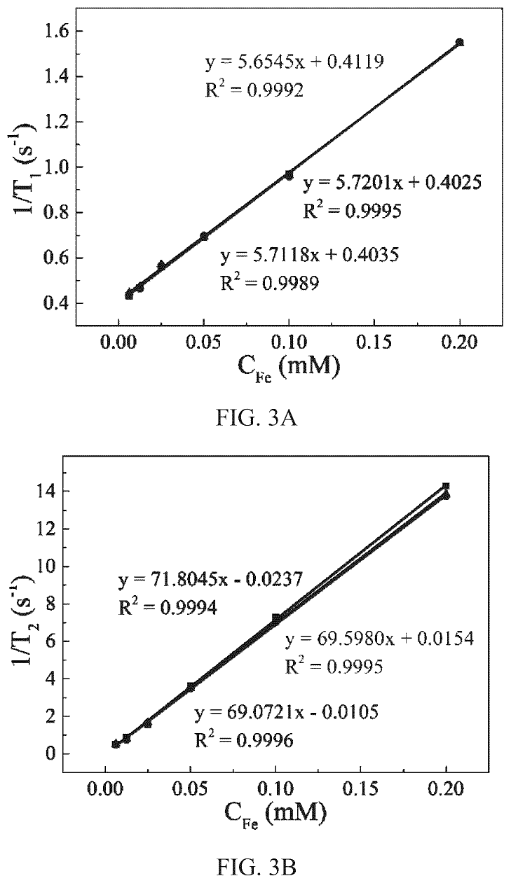

Table 1 shows the r₁ value and the r₂/r₁ ratio of the sample γ-PGA-ES-MION1-8 in Example 1 under different magnetic field intensities (7.0 T, 3.0 T or 1.0 T), and simultaneously shows the r₁ values and the r₂/r₁ ratios of the commercially available products Gadavist and Magnevist under the magnetic field intensity of 3.0 T. The r₁ value of the sample γ-PGA-ES-MION6 in Example 1 under the magnetic field intensity of 3.0 T is 5.7 mM⁻¹ s⁻¹, which is higher than the r₁ value of 4.6 mM⁻¹ s⁻¹ of the commercially available products, thus indicating an excellent magnetic resonance imaging capability. FIG. 4 shows a saturation magnetization diagram of γ-PGA-ES-MION6 in Example 1. It can be seen from the figure that the sample in Example 1 is paramagnetic, which accords with the property of the magnetic ferroferric oxide nanoparticle. A magnitude of the saturation magnetization is positively correlated with the particle size. It can be seen from the figure that the saturation magnetization of the sample in Example 1 is 16 emu/g, which corresponds to the particle size less than 5 nm shown in the electron micrograph in FIG. 1A. The r₂ value of the contrast agent is positively correlated with the saturation magnetization, and small saturation magnetization corresponds to a small r₂ value, which corresponds to the small r₂ value shown in Table 1.

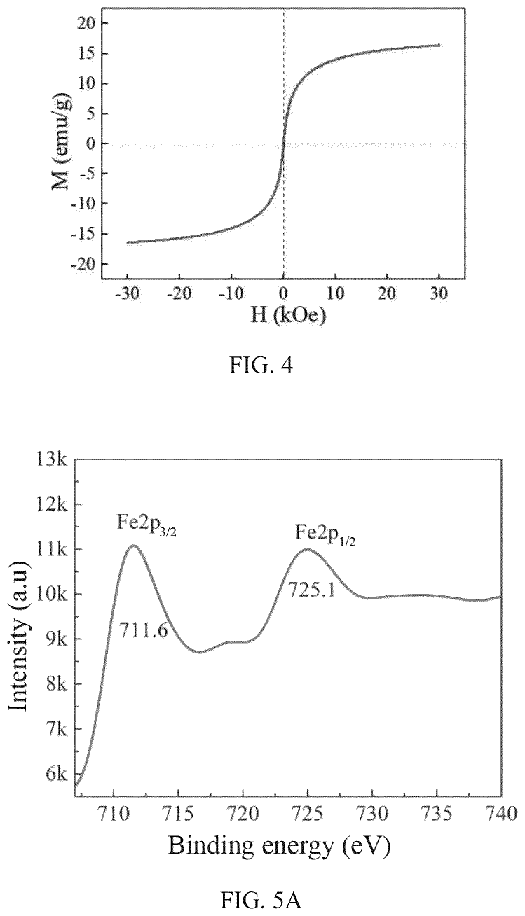

FIG. 5A shows an X-ray photoelectron spectrogram (XPS) of γ-PGA-ES-MION6 in Example 1, and FIG. 5B shows an X-ray diffraction diagram (XRD) of γ-PGA-ES-MION6 in Example 1. Peaks centered at 711.6 eV and 725.1 eV in the XPS of FIG. 5A correspond to binding energies of Fe²⁺2p and Fe³⁺2p respectively, which indicates that trivalent iron and divalent iron exist at the same time, and proves that the sample in Example 1 is the ferroferric oxide. Two characteristic peaks (2θ≈35.6° and 2θ≈62.9°) in the XRD of FIG. 5B correspond to crystal layers [(311) and (440)] of the ferroferric oxide, which proves that the sample in Example 1 is the ferroferric oxide in a crystal structure. The XRD and the XPS jointly prove the successful preparation of the ferroferric oxide in Example 1, and then prove the successful preparation of the magnetic ferroferric oxide nanoparticle in Example 1 in combination with the electron micrograph in FIG. 1B.

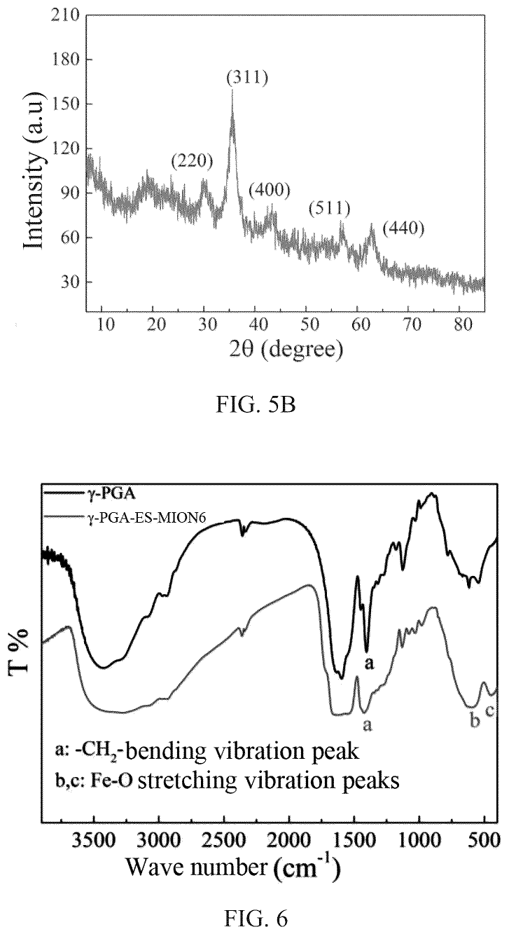

FIG. 6 shows an infrared absorption spectrogram of γ-PGA-ES-MION6 prepared in Example 1. An infrared absorption curve of γ-PGA and an infrared absorption curve of γ-PGA-ES-MION6 both show an absorption peak a (1,404 cm⁻¹), which is a bending vibration peak of —CH₂—, thus indicating the existence of the γ-PGA in the sample. In addition, the infrared absorption curve of the γ-PGA-ES-MION6 shows absorption peaks b and c, which are stretching vibration peaks of Fe—O, while the infrared absorption curve of the γ-PGA does not show the absorption peaks b and c, which further proves the successful preparation of the magnetic ferroferric oxide nanoparticle γ-PGA-ES-MION6.

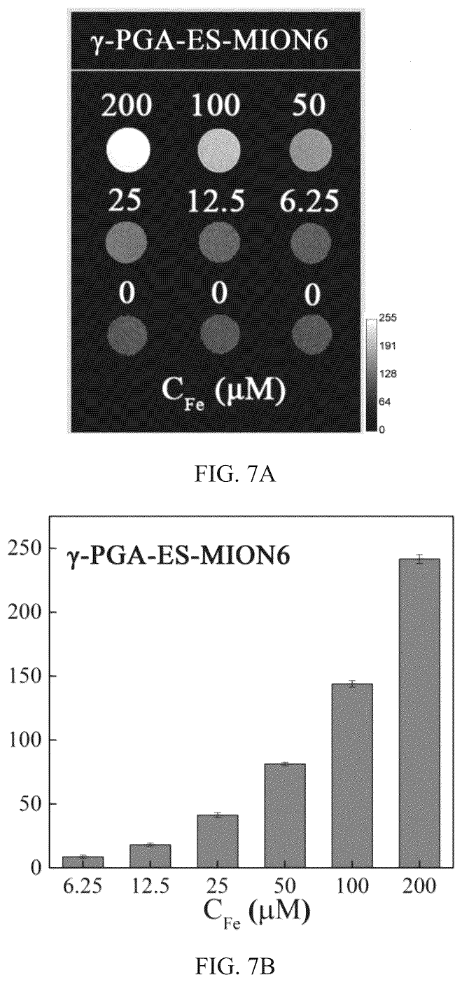

FIG. 7A shows T₁-weighted MRI images of γ-PGA-ES-MION6 aqueous solutions at different concentrations in Example 1, and FIG. 7B shows corresponding MRI signal intensities of γ-PGA-ES-MION6 aqueous solutions at different concentrations in Example 1. The magnetic field intensity is 3.0 T, and scanning parameters are: TE=8.6 ms and TR=500 ms. It can be seen from the MRI image of FIG. 7(a) that an MRI signal of the aqueous solution containing the sample γ-PGA-ES-MION6 is obviously enhanced compared with pure water (a concentration of iron is 0), and shows a gradient enhancement with the increase of the concentration of the sample. It can be seen from FIG. 7(b) that, when the concentration of iron in the γ-PGA-ES-MION6 solution is 200 μM, a signal-to-noise ratio (ΔSNR) of the MRI is 240%, which indicates that the sample γ-PGA-ES-MION6 can effectively improve the contrast and sensitivity of the MRI, thus showing the good magnetic resonance imaging performance of the γ-PGA-ES-MION6 in Example 1.

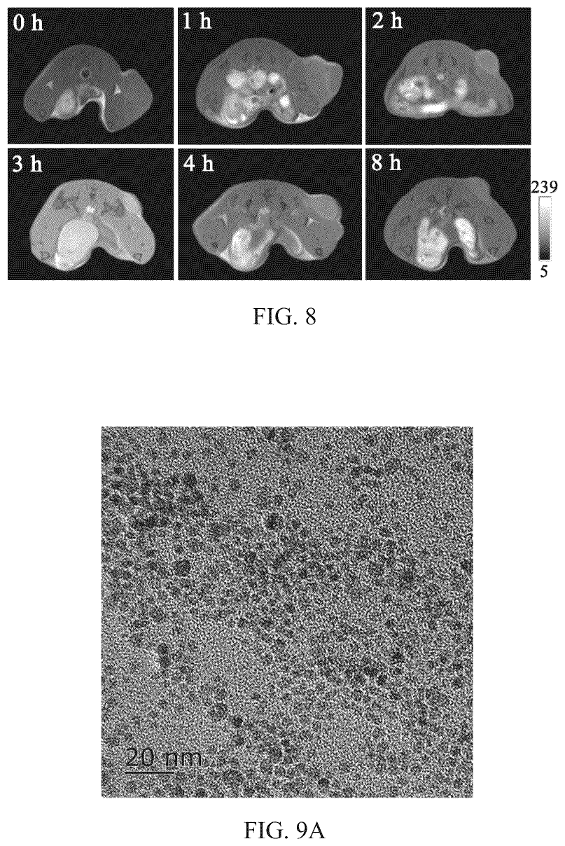

FIG. 8 is a MRI image showing a tumor-bearing mouse after being injected with γ-PGA-ES-MION6 in Example 1, with an injection dosage of 5 mg/kg. 7.0 T magnetic resonance imaging (MRI) is carried out on the mouse at 0 hour, 1 hour, 2 hours, 3 hours, 4 hours and 8 hours before and after tail vein injection of the sample into the mouse. It can be seen from the figure that, with the increase of injection time of the sample, an MRI signal of a tumor shows a trend of increasing first and then decreasing, and the MRI signal is the highest at 3 hours after injection. In-vivo imaging results show that the sample γ-PGA-ES-MION6 in Example 1 has a good magnetic resonance imaging effect on the tumor of the mouse.

### Example 2

### Preparation of Magnetic Ferroferric Oxide Nanoparticle (PASP-ES-MION):

20 mL of polyaspartic acid PASP (M_{w}=7,000 to 8,000) aqueous solution was subjected to bubble deoxygenation with nitrogen in a three-necked flask for 1 hour, and then heated at 100° C. to reflux. 0.4 mL of a mixed aqueous solution containing FeSO₄ and FeCl₃ was added into the flask, and then added with 6 mL of ammonia water. The mixture reacted for 1 hour under magnetic stirring at 100° C., and was allowed to stand and cooled. The sample after the reaction was dialyzed and purified, and the final sample was marked as PASP-ES-MION. Concentrations of the polyaspartic acid aqueous solution, the mixed aqueous solution and the ammonia water and corresponding samples were shown in Table 2.

Physical properties of the sample in Example 2 were characterized. A recovery rate of iron of the sample in Example 2 was calculated to be 89.6%, which indicated that a utilization rate of a raw material was high, so that cost saving was realized. The sample in Example 2 was prepared into 6 aqueous solutions with different concentrations, and in-vitro imaging tests were carried out by a clinical 3.0 T MRI system to obtain longitudinal relaxation time T₁ and transverse relaxation time T₂. A longitudinal magnetic relaxation rate r₁ and a transverse magnetic relaxation rate r₂ were calculated, and results were shown in Table 2.

TABLE 2
Synthesis of PASP-ES-MION and MRI characterization results

| | | | | | Recovery | | | | |
|---|---|---|---|---|---|---|---|---|---|
| Name of | C_{PASP} | C_{FeCl3} | C_{FeSO4} | C_{NH3•H2O} | rate of iron | H₀ | r₁ | r₂ | |
| sample | (mg/mL)^{a} | (mM)^{a} | (mM)^{a} | (%)^{a} | (%)^{b} | (T) | (mM⁻¹ s⁻¹) | (mM⁻¹ s⁻¹) | r₂/r₁ |
| PASP-ES- | 4.0 | 500 | 250 | 1 | 92.7 | 3.0 | 0.99 | 3.71 | 3.76 |
| MION1 | | | | | | | | | |
| PASP-ES- | 2.0 | 500 | 250 | 1 | 96.6 | 3.0 | 2.01 | 14.04 | 6.98 |
| MION2 | | | | | | | | | |
| PASP-ES- | 1 | 500 | 250 | 1 | 89.3 | 3.0 | 4.66 | 88.50 | 19.00 |
| MION3 | | | | | | | | | |
| PASP-ES- | 0.5 | 500 | 250 | 1 | 90.3 | 3.0 | 5.41 | 153.09 | 28.31 |
| MION4 | | | | | | | | | |
| PASP-ES- | 2 | 500 | 250 | 8 | 99.2 | 3.0 | 5.53 ± 0.04 | 31.0 ± 0.05 | 5.62 ± 0.1 |
| MION5 | | | | | | | | | |
| PASP-ES- | 2 | 500 | 250 | 4 | 98.4 | 3.0 | 5.22 ± 0.03 | 27.3 ± 0.56 | 5.22 ± 0.1 |
| MION6 | | | | | | | | | |
| PASP-ES- | 2 | 500 | 250 | 2 | 98.8 | 3.0 | 3.83 ± 0.02 | 25.1 ± 0.51 | 6.54 ± 0.2 |
| MION7 | | | | | | | | | |
| PASP-ES- | 2 | 500 | 250 | 0.5 | 97.5 | 3.0 | 0.32 ± 0.01 | 5.8 ± 0.08 | 18.2 ± 0.4 |
| MION8 | | | | | | | | | |
| PASP-ES- | 1.5 | 375 | 187.5 | 4 | 89.6 | 3.0 | 7.3 | 35.8 | 4.9 |
| MION9 | | | | | | | | | |
| PASP-ES- | 1 | 250 | 125 | 4 | 87.7 | 3.0 | 6.2 | 33.0 | 5.3 |
| MION10 | | | | | | | | | |
| PASP-ES- | 0.5 | 125 | 62.5 | 4 | 97.7 | 3.0 | 6.0 | 38.8 | 6.5 |
| MION11 | | | | | | | | | |

^{a}is a concentration of a reactant added before the reaction; and
^{b}is a molar percentage of an iron content of the ferroferric oxide in the raw material iron added.

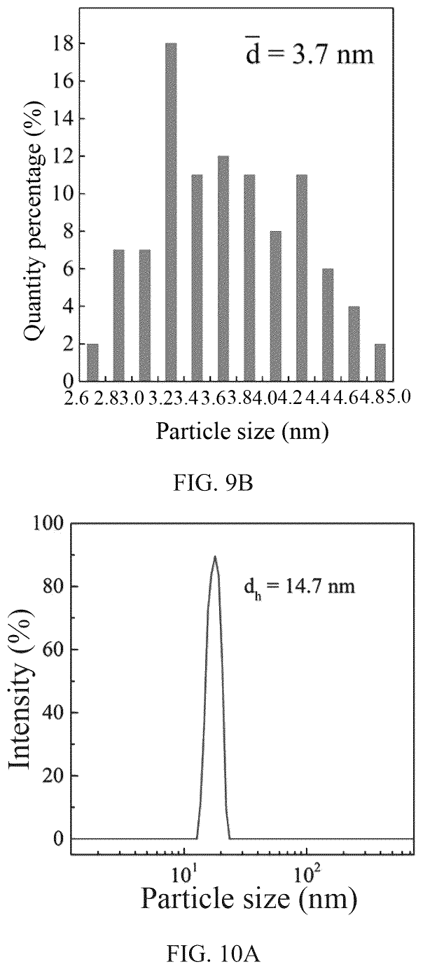

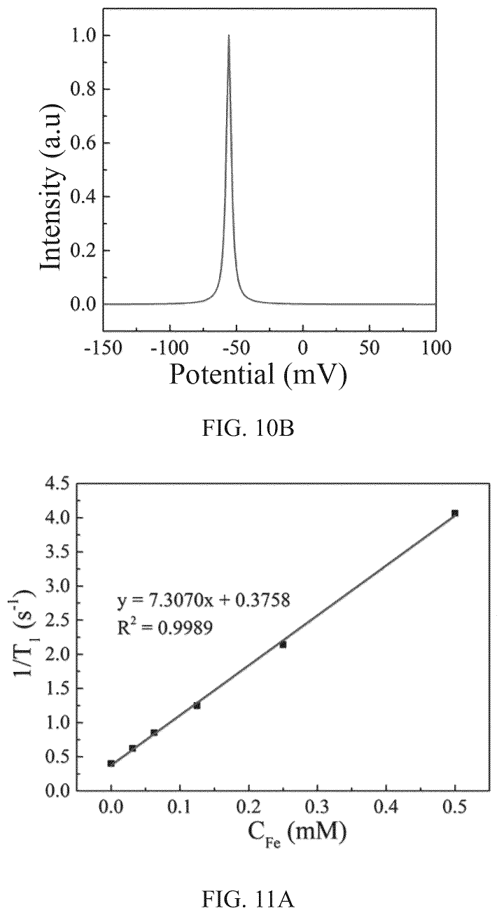

FIG. 9A shows a transmission electron micrograph of the sample PASP-ES-MION9 in Example 2, and FIG. 9B shows a particle size distribution diagram of the sample PASP-ES-MION9 in Example 2. It can be seen from the transmission electron micrograph of FIG. 9A that the nanoparticles are uniform in size and evenly dispersed, and particle size distributions of 100 nanoparticles randomly selected from FIG. 9A are counted to obtain the distribution diagram of FIG. 9 B. It can be seen from the distribution diagram that the particle size of the sample mainly ranges from 2.6 nm to 5.0 nm, and an average particle size of the sample is 3.7 nm, thus meeting the requirement that the particle size of the ferroferric oxide as the T₁ contrast agent is less than 5 nm. FIG. 10A shows a hydraulic diameter of the sample PASP-ES-MION9 in Example 2, and FIG. 10B shows a Zeta potential diagram of the sample PASP-ES-MION9 in Example 2. It is measured that an average value of hydraulic diameters of three samples (PASP-ES-MION9-1, PASP-ES-MION9-2 and PASP-ES-MION9-3) is 14.7 nm, and a PDI of the sample is 0.224, which indicates good dispersity. A Zeta potential of the sample is −50.7 mV, and water phase dispersity of the sample can be improved by charge repulsion, which is beneficial for blood circulation of the sample.

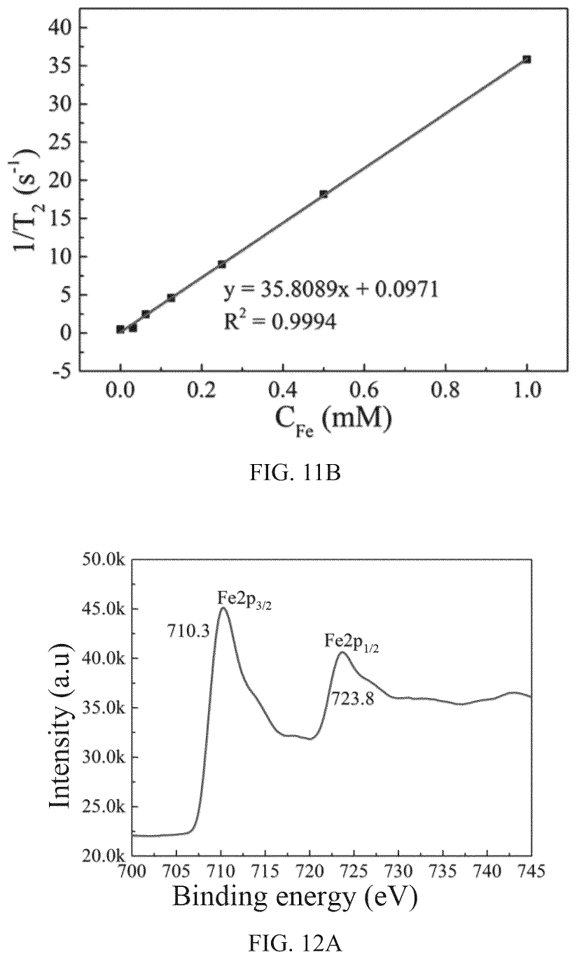

The concentrations of the polyaspartic acid aqueous solution, the mixed aqueous solution and the ammonia water have a great influence on the MRI performance of the sample. Under different synthetic conditions, the sample PASP-ES-MION9-10 has a better comprehensive MRI performance. Taking the PASP-ES-MION9 as an example, FIG. 11A and FIG. 11B show MRI relaxation rates of the sample PASP-ES-MION9 in Example 2. It can be seen from FIG. 11A or FIG. 11B that goodness of linear fitting of a fitted line is 0.99 or more, which proves that the sample has a good linear relationship. Table 2 shows the r₁ value and the r₂/r₁ ratio of the sample PASP-ES-MION9 in Example 2 under the magnetic field intensity of 3.0 T. The r₁ value of the sample PASP-ES-MION9 in Example 2 under the magnetic field intensity of 3.0 T is 7.3 mM⁻¹ s⁻¹, which is higher than the r₁ value of 4.64 mM⁻¹ s⁻¹ of the commercially available products. In addition, the r₂/r₁ ratio of the sample PASP-ES-MION9 under the magnetic field intensity of 3.0 T is only 4.9. The results show that the sample PASP-ES-MION9 in Example 2 under the magnetic field intensity of 3.0 T has an extremely high r₁ value and an extremely low r₂/r₁ ratio.

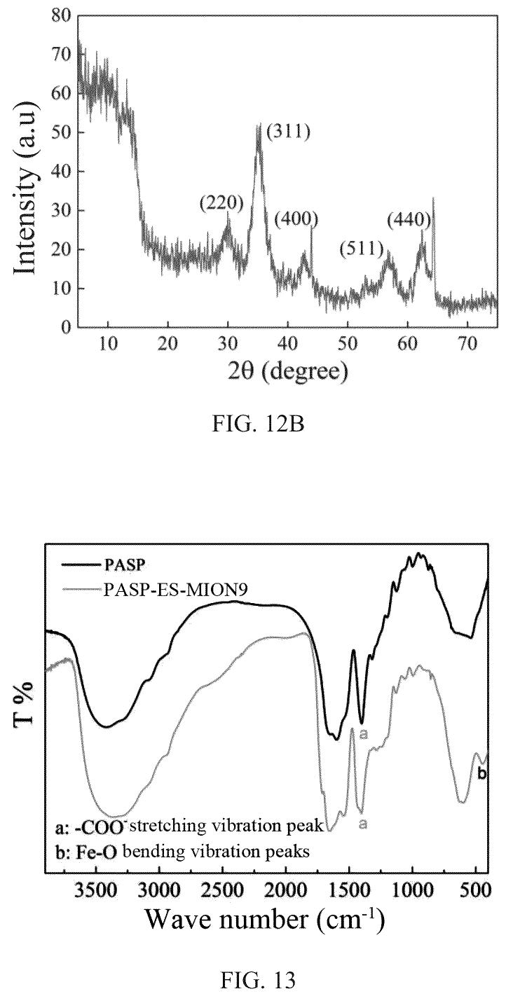

FIG. 12A shows an X-ray photoelectron spectrogram of the PASP-ES-MION9 in Example 2, and FIG. 12B shows an X-ray diffraction diagram of the PASP-ES-MION9 in Example 2. Peaks centered at 710.3 eV and 723.8 eV in the XPS of FIG. 12A correspond to binding energies of Fe²⁺2p and Fe³⁺2p respectively, which indicates that trivalent iron and divalent iron exist at the same time, and proves that the sample in Example 2 is the ferroferric oxide. Two characteristic peaks (2θ≈35.3° and 2θ≈62.4°) in the XRD of FIG. 12B correspond to crystal layers [(311) and (440)] of the ferroferric oxide, which proves that the sample in Example 2 is the ferroferric oxide in a crystal structure. The XRD and the XPS jointly prove the successful preparation of the ferroferric oxide in Example 2, and then prove the successful preparation of the magnetic ferroferric oxide nanoparticle in Example 2 in combination with the electron micrograph in FIG. 9A.

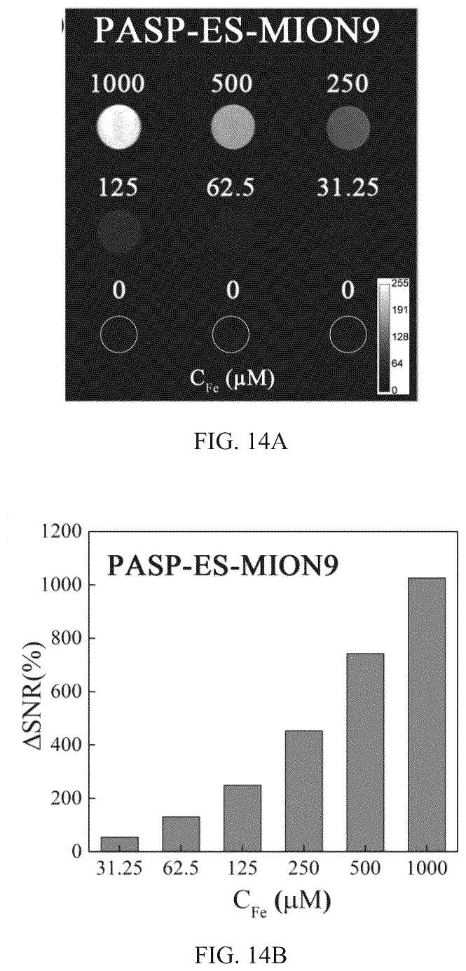

FIG. 13 shows an infrared absorption spectrogram of PASP-ES-MION9 prepared in Example 2. An infrared absorption curve of PASP and an infrared absorption curve of PASP-ES-MION9 both show an absorption peak a (1,400 cm⁻¹), which is a stretching vibration peak of —COO—, thus indicating the existence of the PASP in the sample. In addition, the infrared absorption curve of PASP-ES-MION9 shows an absorption peak b, which is a stretching vibration peak of Fe—O, while the infrared absorption curve of the PASP does not show the absorption peak b, which further proves the successful preparation of the magnetic ferroferric oxide nanoparticle PASP-ES-MION9.

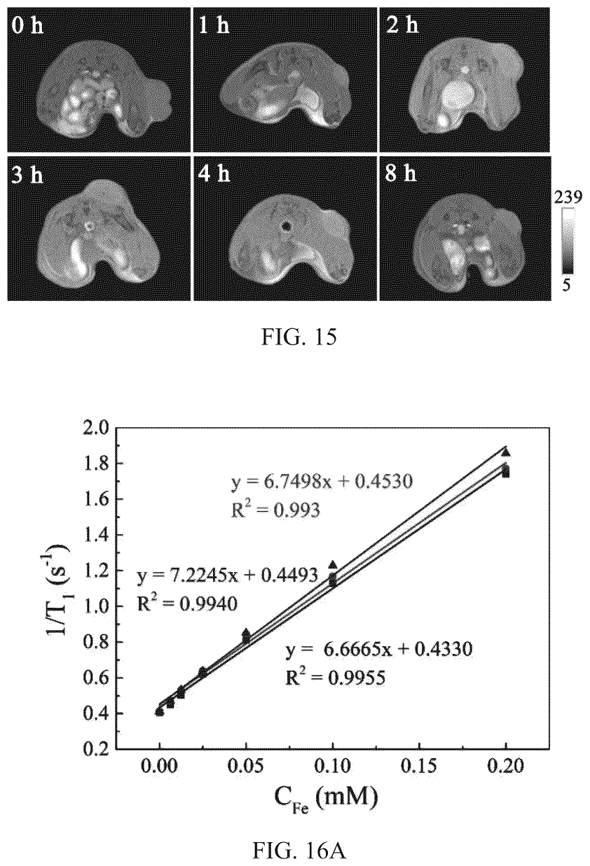

FIG. 14A shows T₁-weighted MRI images of PASP-ES-MION9 aqueous solutions at different concentrations in Example 2, and FIG. 14B shows corresponding MRI signal intensities of PASP-ES-MION9 aqueous solutions at different concentrations in Example 2. The magnetic field intensity is 3.0 T, and scanning parameters are: TE=8.4 ms and TR=200 ms. It can be seen from the MRI image of FIG. 14 (a) that an MRI signal of the aqueous solution containing the sample PASP-ES-MION9 is obviously enhanced compared with pure water (a concentration of iron is 0), and shows a gradient enhancement with the increase of the concentration of the sample. It can be seen from FIG. 14 (b) that, when the concentration of iron in the sample PASP-ES-MION9 aqueous solution is 1,000 μM, ΔSNR of the MRI is 1,025%, which proves that the PASP-ES-MION9 can effectively improve the contrast and sensitivity of the MRI, thus showing the excellent magnetic resonance imaging performance of the PASP-ES-MION9 in Example 2.

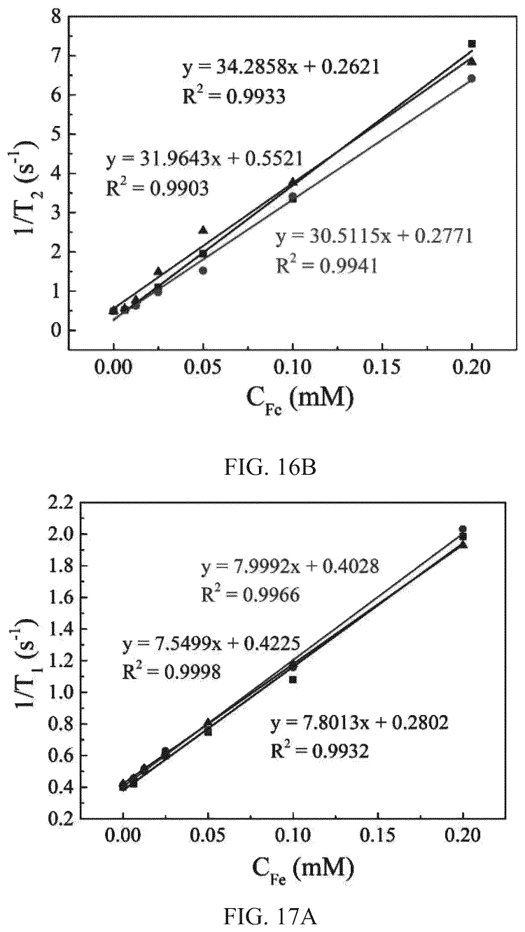

FIG. 15 shows a MRI image showing a tumor-bearing mouse after being injected with PASP-ES-MION9 in Example 2, with an injection dosage of 5 mg/kg. 7.0 T magnetic resonance imaging (MRI) is carried out on the mouse at 0 hour, 1 hour, 2 hours, 3 hours, 4 hours and 8 hours before and after tail vein injection of the sample into the mouse. It can be seen from the figure that, with the increase of injection time of the sample, an MRI signal of a tumor is increased first and then decreased, and the MRI signal is the highest at 3 hours after injection. In-vivo imaging results show that the PASP-ES-MION9 in Example 2 has a good magnetic resonance imaging effect on the tumor of the mouse.

### Example 3

### Preparation of Magnetic Ferroferric Oxide Nanoparticle (HPMA-ES-MION):

20 mL of 2 mg/mL polymaleic acid HPMA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as HPMA-ES-MION.

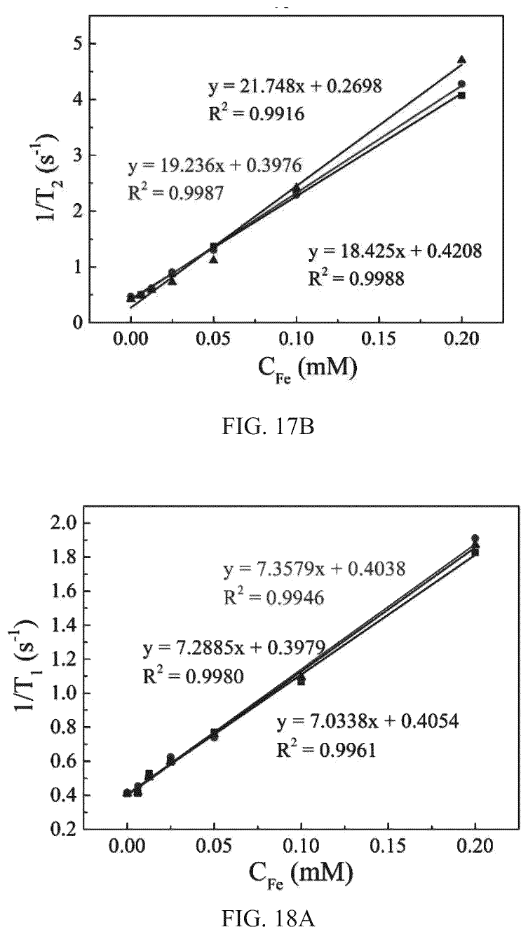

A performance test was carried out on the sample in Example 3. A recovery rate of iron of the sample in Example 3 was calculated to be 87.2%, which indicated that a utilization rate of a raw material was high, so that cost saving was realized. The sample in Example 3 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples HPMA-ES-MION-1, HPMA-ES-MION-2 and HPMA-ES-MION-3 were tested in parallel for three times). As shown in FIG. 16A and FIG. 16B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.9±0.3 mM⁻¹ s⁻¹, an r₂ value was 32.3±1.9 mM⁻¹ s⁻¹, and an r₂/r₁ ratio was 4.7±0.4 in Example 3. It was proved that the magnetic ferroferric oxide nanoparticle (HPMA-ES-MION) in Example 3 had a high r₁ value and a low r₂/r₁ ratio, and could be used as a T₁-weighted MRI contrast agent with good biocompatibility.

### Example 4

### Preparation of Magnetic Ferroferric Oxide Nanoparticle (PEAA-ES-MION):

20 mL of 2 mg/mL poly(2-ethylacrylic acid) PEAA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PEAA-ES-MION.

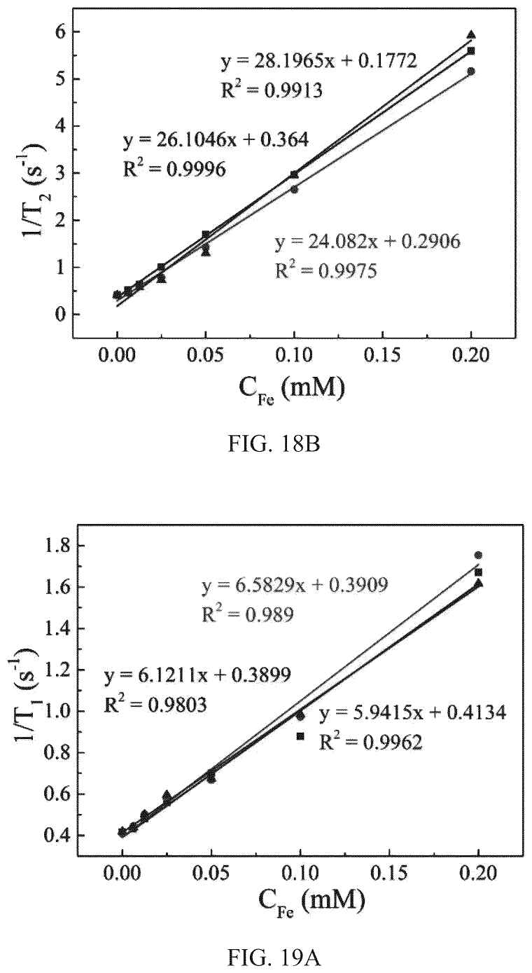

A performance test was carried out on the sample in Example 4. A recovery rate of iron of the sample in Example 4 was calculated to be 89.1%, which indicated that a utilization rate of a raw material was high, so that cost saving was realized. The sample in Example 4 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PEAA-ES-MION-1, PEAA-ES-MION-2 and PEAA-ES-MION-3 were tested in parallel for three times). As shown in FIG. 17 A and FIG. 17B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.8±0.2 mM⁻¹ s⁻¹, an r₂ value was 19.8±1.7 mM⁻¹ s⁻¹, and an r₂/r₁ ratio was 2.5±0.3 in Example 4. The results proved that the magnetic ferroferric oxide nanoparticle (PEAA-ES-MION) in Example 4 had an extremely high r₁ value and a low r₂/r₁ ratio, and could be used as a T₁-weighted MRI contrast agent with good biocompatibility.

### Example 5

### Preparation of Magnetic Ferroferric Oxide Nanoparticle (PESA-ES-MION):

20 mL of 2 mg/mL polyepoxysuccinic acid PESA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PESA-ES-MION.

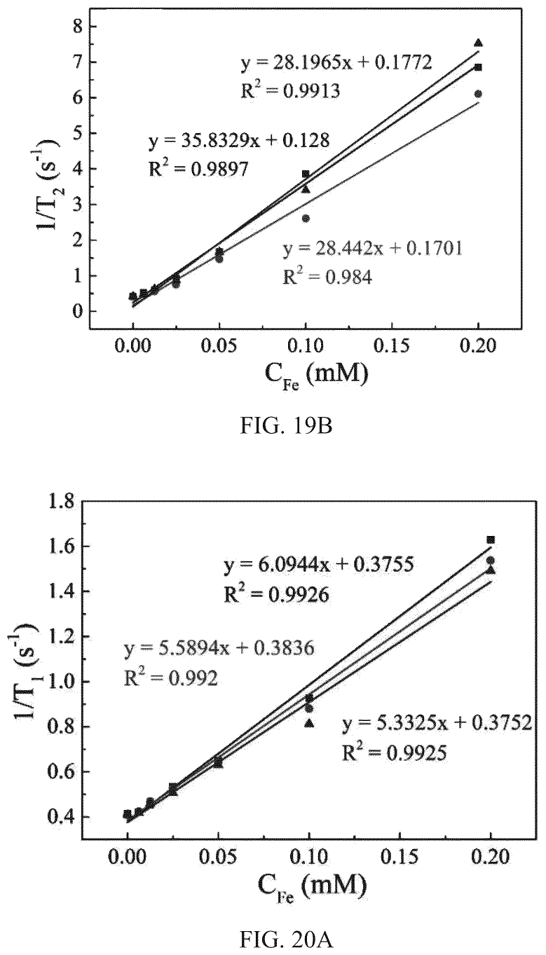

A performance test was carried out on the sample in Example 5. A recovery rate of iron of the sample in Example 5 was calculated to be 93.2%, which indicated that a utilization rate of a raw material was high, so that cost saving was realized. The sample in Example 5 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PESA-ES-MION-1, PESA-ES-MION-2 and PESA-ES-MION-3 were tested in parallel for three times). As shown in FIG. 18A and FIG. 18B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.2±0.2 mM⁻¹ s⁻¹, an r₂ value was 26.1±2.1 mM⁻¹ s⁻¹, and an r₂/r₁ ratio was 3.6±0.3 in Example 5. The results proved that the ferroferric oxide nanoparticle (PESA-ES-MION) in Example 5 had an extremely high r₁ value and a low r₂/r₁ ratio, and could be used as a T₁-weighted MRI contrast agent with good biocompatibility.

### Example 6

### Preparation of Magnetic Ferroferric Oxide Nanoparticle (ε-PL-ES-MION):

20 mL of 2 mg/mL polylysine ε-PL (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as ε-PL-ES-MION.

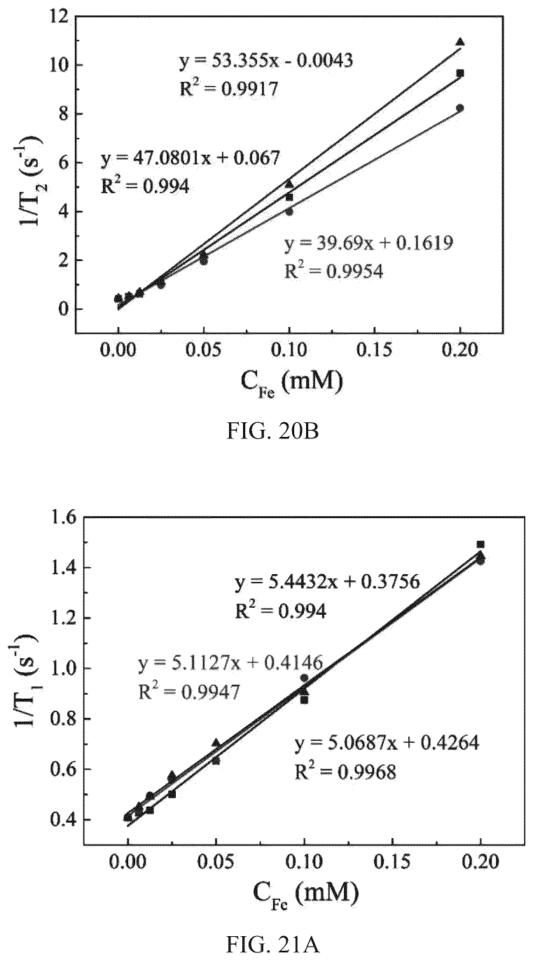

A performance test was carried out on the sample in Example 6. A recovery rate of iron of the sample in Example 6 was calculated to be 82.6%, which indicated that a utilization rate of a raw material was high, so that cost saving was realized. The sample in Example 6 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples ε-PL-ES-MION-1, ε-PL-ES-MION-2 and ε-PL-ES-MION-3 were tested in parallel for three times). As shown in FIG. 19A and FIG. 19B, a change relationship between 1/Tᵢ and a concentration of iron was drawn, and a slope of a fitted line was a relaxation rate of Tᵢ (i=1 or 2). Thus, it could be seen that, under the 3.0 T MRI system, an r₁ value was 6.2±0.3 mM⁻¹ s⁻¹, an r₂ value was 32.5±3.8 mM⁻¹ s⁻¹, and an r₂/r₁ ratio was 5.3±0.9 in Example 6. The results proved that the magnetic ferroferric oxide nanoparticle (ε-PL-ES-MION) in Example 6 had an extremely high r₁ value and a low r₂/r₁ ratio, and could be used as a T₁-weighted MRI contrast agent with good biocompatibility.

### Example 7

### Preparation of Magnetic Ferroferric Oxide Nanoparticle (PLH-ES-MION):

20 mL of 2 mg/mL polyhistidine PLH (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PLH-ES-MION.

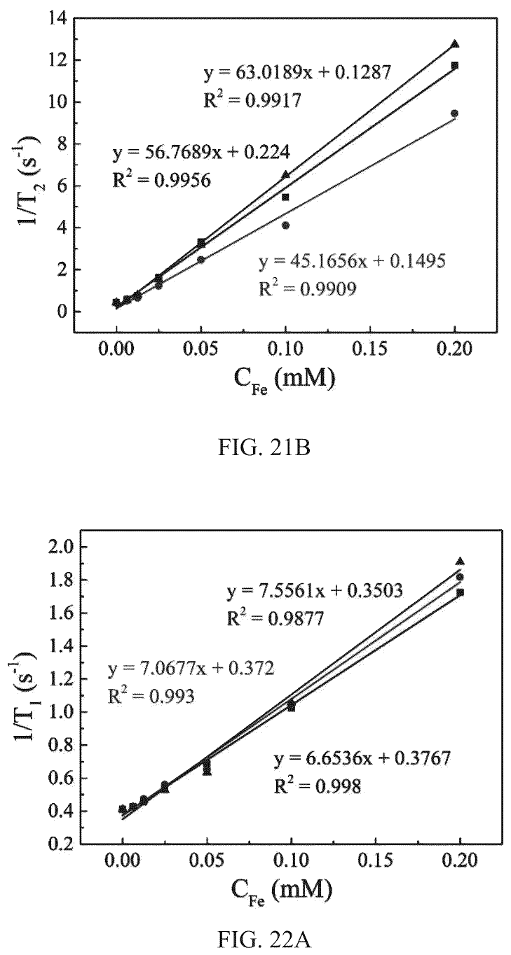

A performance test was carried out on the sample in Example 6. A recovery rate of iron of the sample in Example 7 was calculated to be 85.7%, which indicated that a utilization rate of a raw material was high, so that cost saving was realized. The sample in Example 7 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PLH-ES-MION-1, PLH-ES-MION-2 and PLH-ES-MION-3 were tested in parallel for three times). As shown in FIG. 20 A and FIG. 20B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 5.7±0.4 mM⁻¹ s⁻¹, an r₂ value was 46.6±7.0 mM⁻¹ s⁻¹, and an r₂/r₁ ratio was 8.3±1.5 in Example 7. The results proved that the magnetic ferroferric oxide nanoparticle (PLH-ES-MION) in Example 7 had an extremely high r₁ value and a low r₂/r₁ ratio, and could be used as a T₁-weighted MRI contrast agent with good biocompatibility.

### Example 8

### Preparation of Magnetic Ferroferric Oxide Nanoparticle (PLR-ES-MION):

20 mL of 2 mg/mL poly-L-arginine PLR (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PLR-ES-MION.

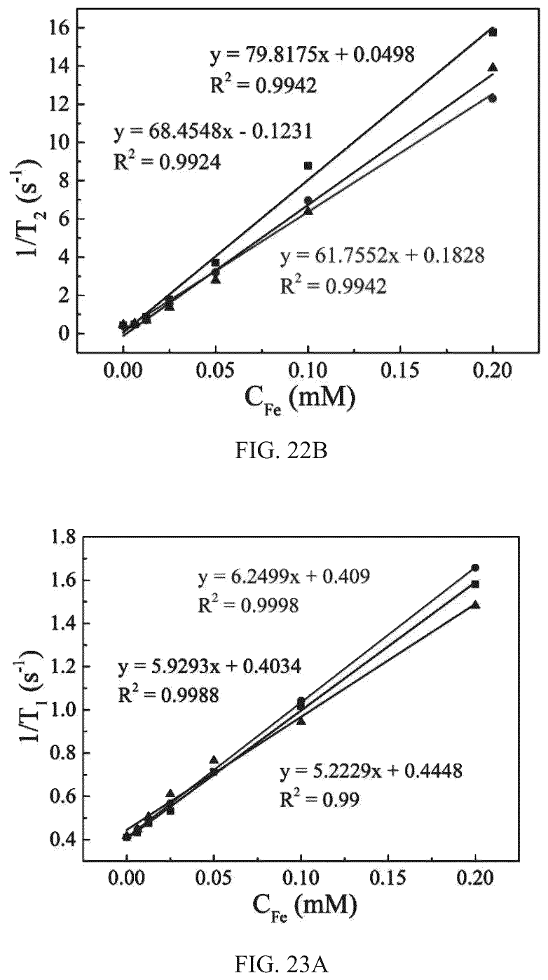

A performance test was carried out on the sample in Example 8. A recovery rate of iron of the sample in Example 8 was calculated to be 83.9%, which indicated that a utilization rate of a raw material was high, so that cost saving was realized. The sample in Example 8 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PLR-ES-MION-1, PLR-ES-MION-2 and PLR-ES-MION-3 were tested in parallel for three times). As shown in FIG. 21 A and FIG. 21B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 5.2±0.2 mM⁻¹ s⁻¹, an r₂ value was 55.0±9.1 mM⁻¹ s⁻¹, and an r₂/r₁ ratio was 10.6±1.8 in Example 8. The results proved that the magnetic ferroferric oxide nanoparticle (PLR-ES-MION) in Example 8 had an extremely high r₁ value and a low r₂/r₁ ratio, and could be used as a T₁-weighted MRI contrast agent with good biocompatibility.

### Example 9

### Preparation of Magnetic Ferroferric Oxide Nanoparticle (PDDA-ES-MION):

20 mL of 2 mg/mL polydimethyl diallyl ammonium chloride PDDA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PDDA-ES-MION.

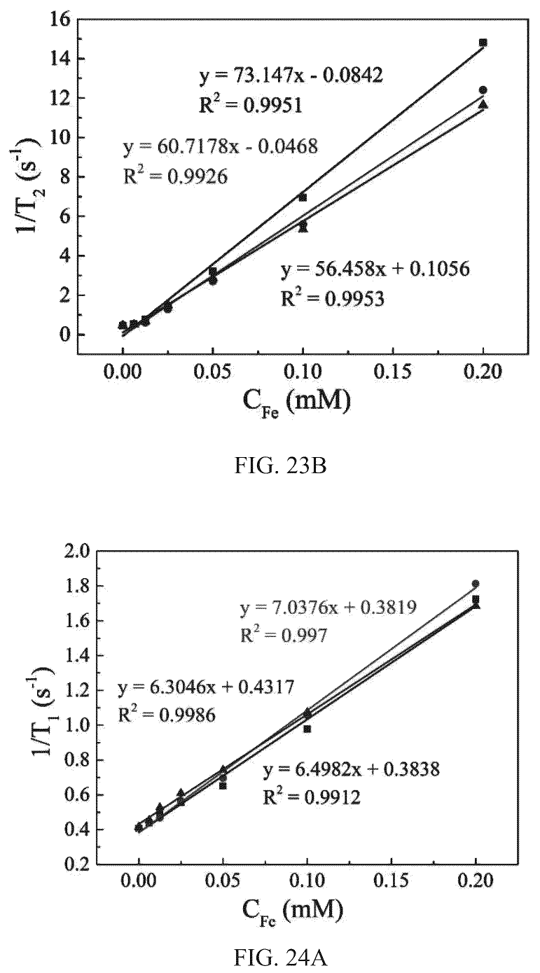

A performance test was carried out on the sample in Example 9. A recovery rate of iron of the sample in Example 9 was calculated to be 90.4%, which indicated that a utilization rate of a raw material was high, so that cost saving was realized. The sample in Example 9 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PDDA-ES-MION-1, PDDA-ES-MION-2 and PDDA-ES-MION-3 were tested in parallel for three times). As shown in FIG. 22 A and FIG. 22B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.1±0.5 mM⁻¹ s⁻¹, an r₂ value was 70.0±9.1 mM⁻¹ s⁻¹, and an r₂/r₁ ratio was 9.9±1.8 in Example 9. The results proved that the magnetic ferroferric oxide nanoparticle (PDDA-ES-MION) in Example 9 had a high r₁ value and a low r₂/r₁ ratio, and could be used as a T₁-weighted MRI contrast agent with good biocompatibility.

### Example 10

### Preparation of Magnetic Ferroferric Oxide Nanoparticle (PSer-ES-MION):

20 mL of 2 mg/mL polyserine PSer (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PSer-ES-MION.

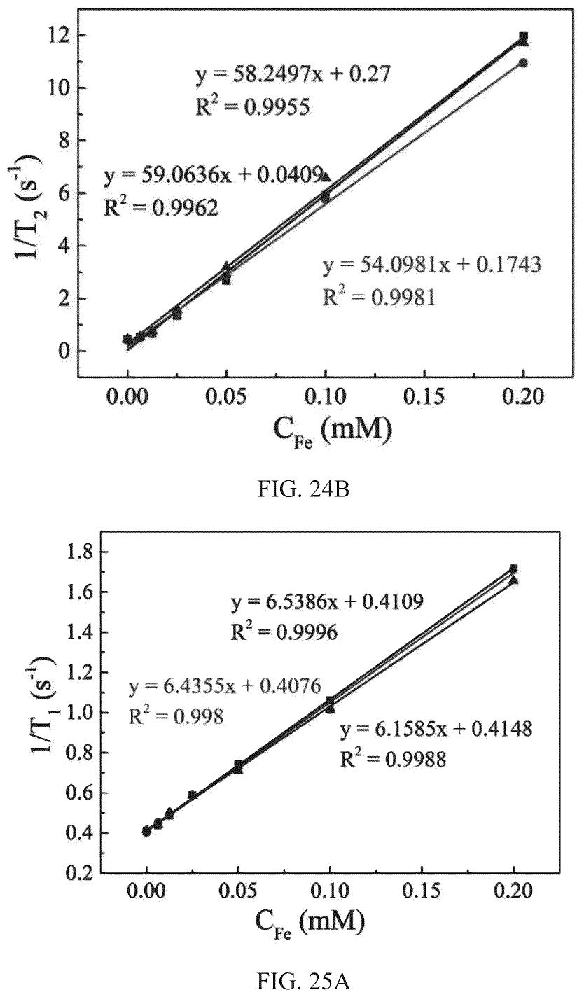

A performance test was carried out on the sample in Example 10. A recovery rate of iron of the sample in Example 10 was calculated to be 88.2%, which indicated that a utilization rate of a raw material was high, so that cost saving was realized. The sample in Example 10 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PSer-ES-MION-1, PSer-ES-MION-2 and PSer-ES-MION-3 were tested in parallel for three times). As shown in FIG. 23 A and FIG. 23B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 5.8±0.25 mM⁻¹ s⁻¹, an r₂ value was 63.4±8.7 mM⁻¹ s⁻¹, and an r₂/r₁ ratio was 11.0±1.3 in Example 10. The results proved that the magnetic ferroferric oxide nanoparticle (PSer-ES-MION) in Example 10 had a high r₁ value and a low r₂/r₁ ratio, and could be used as a T₁-weighted MRI contrast agent with good biocompatibility.

### Example 11

### Preparation of Magnetic Ferroferric Oxide Nanoparticle (PThr-ES-MION):

20 mL of 2 mg/mL polythreonine PThr (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PThr-ES-MION.

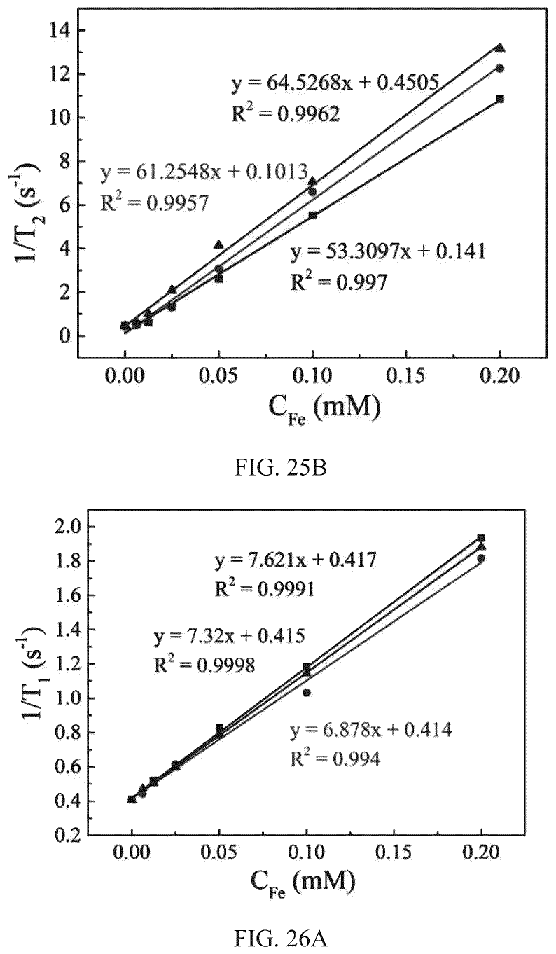

A performance test was carried out on the sample in Example 11. A recovery rate of iron of the sample in Example 11 was calculated to be 85.7%, which indicated that a utilization rate of a raw material was high, so that cost saving was realized. The sample in Example 11 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PThr-ES-MION-1, PThr-ES-MION-2 and PThr-ES-MION-3 were tested in parallel for three times). As shown in FIG. 24A and FIG. 24B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.6±0.4 mM⁻¹ s⁻¹, an r₂ value was 57.2±2.5 mM⁻¹ s⁻¹, and an r₂/r₁ ratio was 8.7±0.8 in Example 11. The results proved that the magnetic ferroferric oxide nanoparticle (PThr-ES-MION) in Example 11 had a high r₁ value and a low r₂/r₁ ratio, and could be used as a T₁-weighted MRI contrast agent with good biocompatibility.

### Example 12

### Preparation of Magnetic Ferroferric Oxide Nanoparticle (PTyr-ES-MION):

20 mL of 2 mg/mL polytyrosine PTyr (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PTyr-ES-MION.

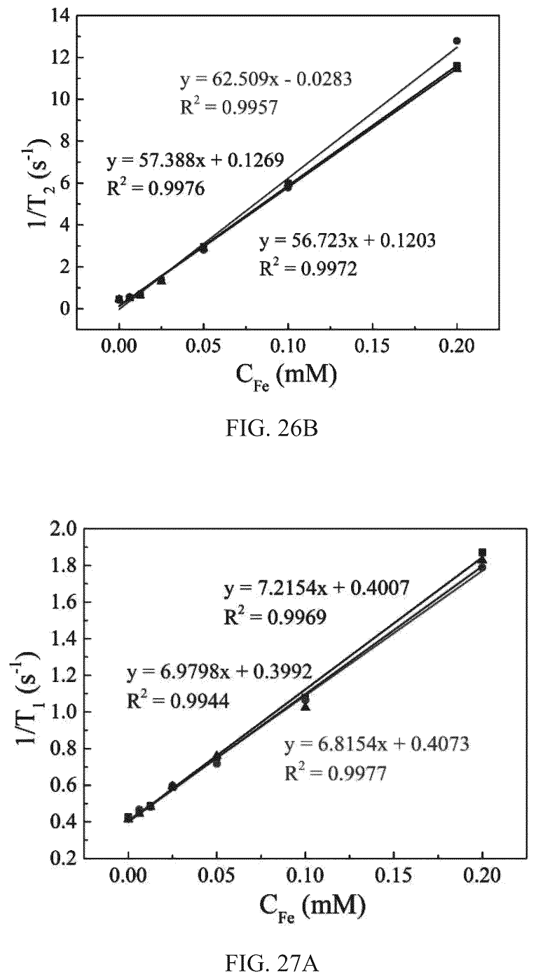

A performance test was carried out on the sample in Example 12. A recovery rate of iron of the sample in Example 12 was calculated to be 83.2%, which indicated that a utilization rate of a raw material was high, so that cost saving was realized. The sample in Example 12 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PTyr-ES-MION-1, PTyr-ES-MION-2 and PTyr-ES-MION-3 were tested in parallel for three times). As shown in FIG. 25A and FIG. 25B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.4±0.2 mM⁻¹ s⁻¹, an r₂ value was 59.7±5.8 mM⁻¹ s⁻¹, and an r₂/r₁ ratio was 9.4±1.2 in Example 12. The results proved that the magnetic ferroferric oxide nanoparticle (Pyr-ES-MION) in Example 12 had a high r₁ value and a low r₂/r₁ ratio, and could be used as a T₁-weighted MRI contrast agent with good biocompatibility.

### Example 13

### Preparation of Magnetic Ferroferric Oxide Nanoparticle (TA-ES-MION):

20 mL of 2 mg/mL tannic acid TA (M_{w}=1,700) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as TA-ES-MION.

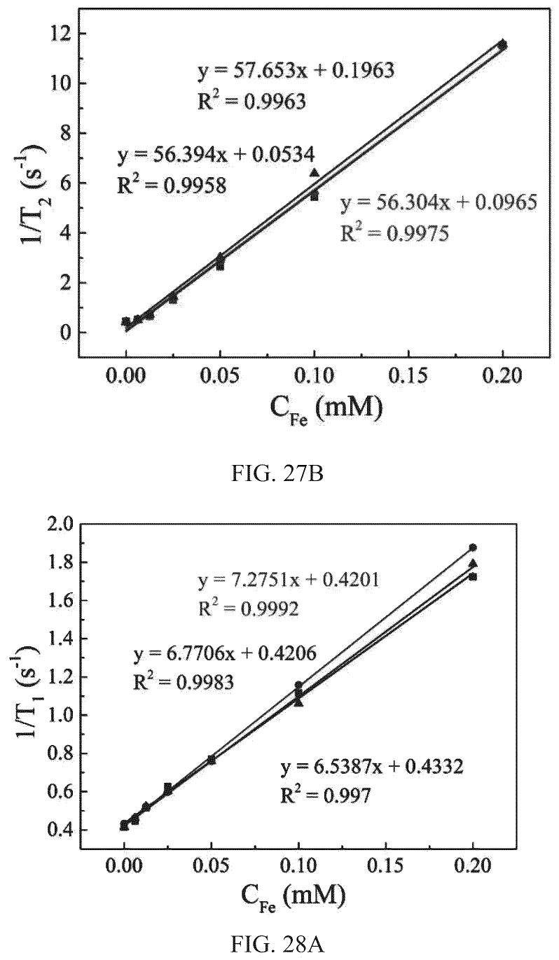

A performance test was carried out on the sample in Example 13. A recovery rate of iron of the sample in Example 13 was calculated to be 92.7%, which indicated that a utilization rate of a raw material was high, so that cost saving was realized. The sample in Example 13 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples TA-ES-MION-1, TA-ES-MION-2 and TA-ES-MION-3 were tested in parallel for three times). As shown in FIG. 26A and FIG. 26B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.3±0.4 mM⁻¹ s⁻¹, an r₂ value was 58.9±3.2 mM⁻¹ s⁻¹, and an r₂/r₁ ratio was 8.1±0.8 in Example 13. The results proved that the magnetic ferroferric oxide nanoparticle (TA-ES-MION) in Example 13 had a high r₁ value and a low r₂/r₁ ratio, and could be used as a T₁-weighted MRI contrast agent with good biocompatibility.

### Example 14

### Preparation of Magnetic Ferroferric Oxide Nanoparticle (PolyQ-ES-MION):

20 mL of 2 mg/mL polyglutamine PolyQ (M_{w=}2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PolyQ-ES-MION.

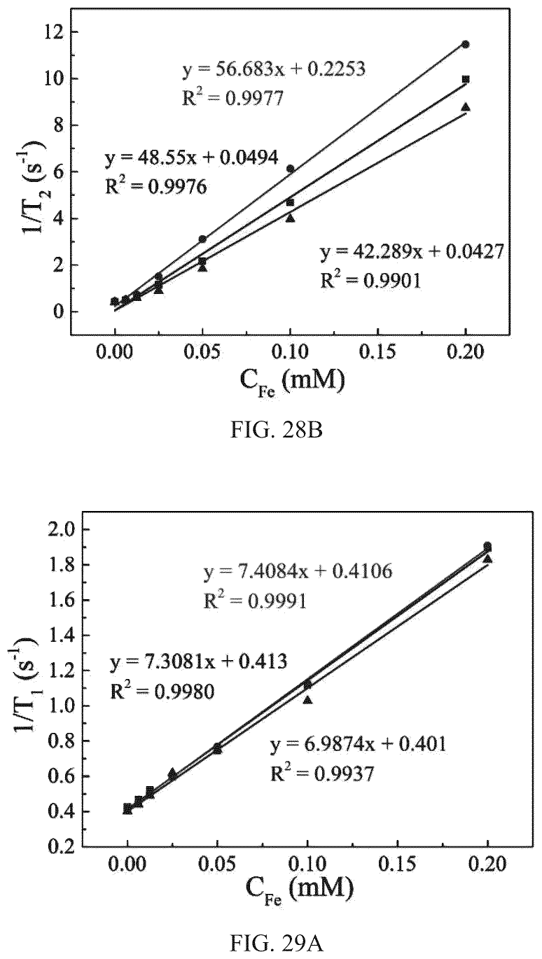

A performance test was carried out on the sample in Example 14. A recovery rate of iron of the sample in Example 14 was calculated to be 87.5%, which indicated that a utilization rate of a raw material was high, so that cost saving was realized. The sample in Example 14 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PolyQ-ES-MION-1, PolyQ-ES-MION-2 and PolyQ-ES-MION-3 were tested in parallel for three times). As shown in FIG. 27 A and FIG. 27B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.0±0.2 mM⁻¹ s⁻¹, an r₂ value was 56.8±0.8 mM⁻¹ s⁻¹, and an r₂/r₁ ratio was 8.1±0.3 in Example 14. The results proved that the magnetic ferroferric oxide nanoparticle (PolyQ-ES-MION) in Example 14 had a high r₁ value and a low r₂/r₁ ratio, and could be used as a T₁-weighted MRI contrast agent with good biocompatibility.

### Example 15

### Preparation of Magnetic Ferroferric Oxide Nanoparticle (PHEA-ES-MION):

20 mL of 2 mg/mL polyasparamide PHEA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PHEA-ES-MION.

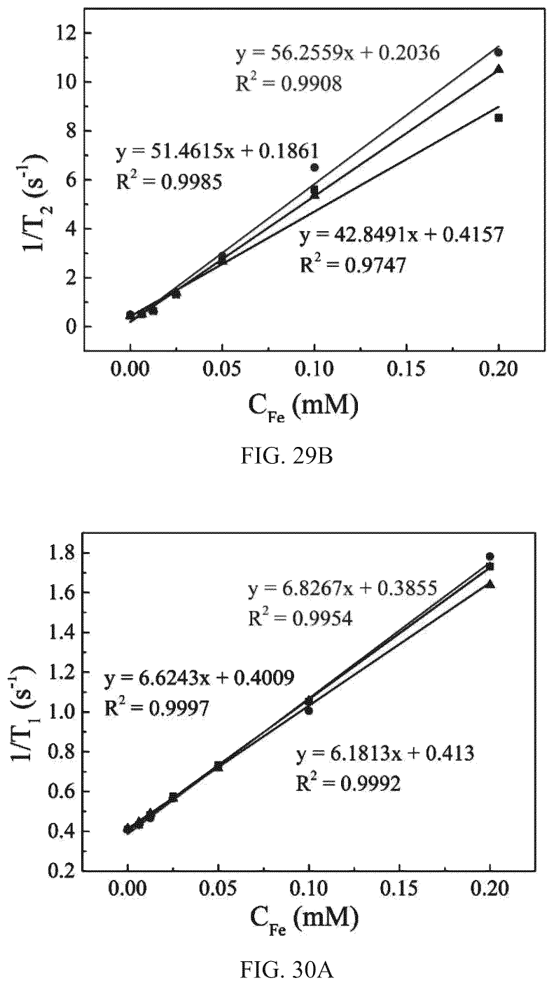

A performance test was carried out on the sample in Example 15. A recovery rate of iron of the sample in Example 15 was calculated to be 85.4%, which indicated that a utilization rate of a raw material was high, so that cost saving was realized. The sample in Example 15 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PHEA-ES-MION-1, PHEA-ES-MION-2 and PHEA-ES-MION-3 were tested in parallel for three times). As shown in FIG. 28A and FIG. 28B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.9±0.4 mM⁻¹ s⁻¹, an r₂ value was 49.2±7.2 mM⁻¹ s⁻¹, and an r₂/r₁ ratio was 7.2±0.8 in Example 15. The results proved that the magnetic ferroferric oxide nanoparticle (PHEA-ES-MION) in Example 15 had a high r₁ value and a low r₂/r₁ ratio, and could be used as a T₁-weighted MRI contrast agent with good biocompatibility.

### Example 16

### Preparation of Magnetic Ferroferric Oxide Nanoparticle (PAM-ES-MION):

20 mL of 2 mg/mL polyacrylamide PAM (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PAM-ES-MION.

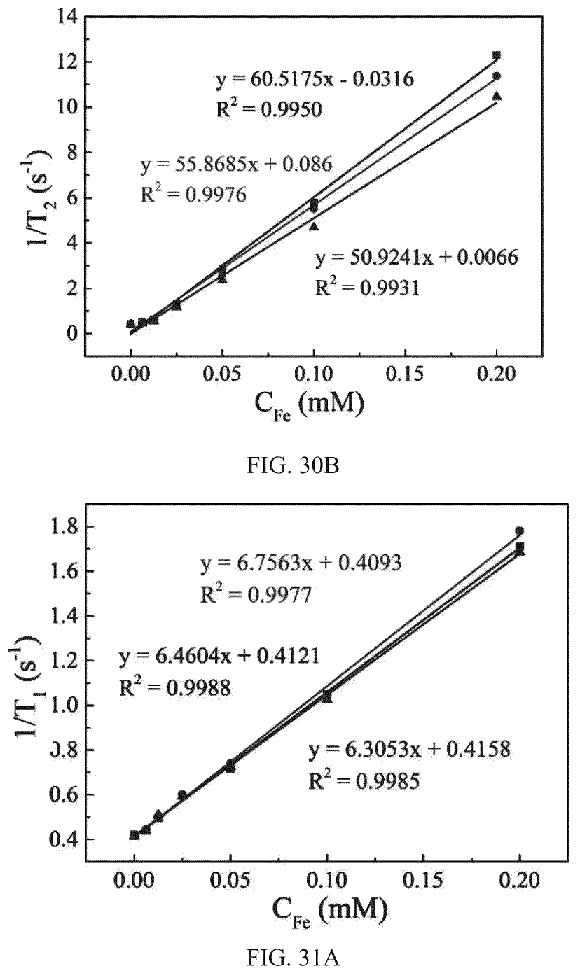

A performance test was carried out on the sample in Example 16. A recovery rate of iron of the sample in Example 16 was calculated to be 84.3%, which indicated that a utilization rate of a raw material was high, so that cost saving was realized. The sample in Example 16 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PAM-ES-MION-1, PAM-ES-MION-2 and PAM-ES-MION-3 were tested in parallel for three times). As shown in FIG. 29A and FIG. 29B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.2±0.2 mM⁻¹ s⁻¹, an r₂ value was 50.2±6.8 mM⁻¹ s⁻¹, and an r₂/r₁ ratio was 6.9±0.9 in Example 16. The results proved that the magnetic ferroferric oxide nanoparticle (PAM-ES-MION) in Example 16 had a high r₁ value and a low r₂/r₁ ratio, and could be used as a T₁-weighted MRI contrast agent with good biocompatibility.

### Example 17

### Preparation of Magnetic Ferroferric Oxide Nanoparticle (PMAM-ES-MION):

20 mL of 2 mg/mL polymethacrylamide PMAM (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as PMAM-ES-MION.

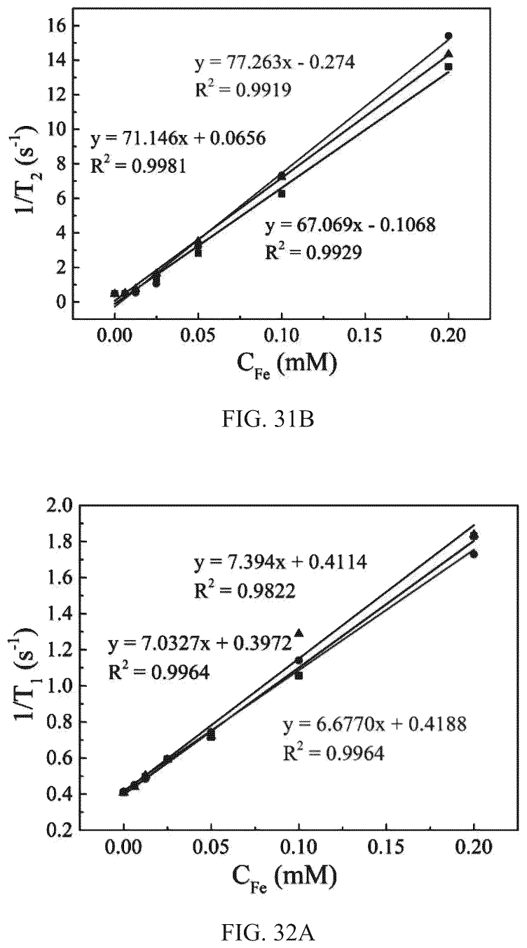

A performance test was carried out on the sample in Example 17. A recovery rate of iron of the sample in Example 17 was calculated to be 90.7%, which indicated that a utilization rate of a raw material was high, so that cost saving was realized. The sample in Example 17 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples PMAM-ES-MION-1, PMAM-ES-MION-2 and PMAM-ES-MION-3 were tested in parallel for three times). As shown in FIG. 30A and FIG. 30B, a change relationship between 1/T; and a concentration of iron was drawn, and a slope of a fitted line was a magnetic relaxation rate of Tᵢ (i=1 or 2). Thus, it could be seen that, under the 3.0 T MRI system, an r₁ value was 6.5±0.3 mM⁻¹ s⁻¹, an r₂ value was 55.8±4.8 mM⁻¹ s⁻¹, and an r₂/r₁ ratio was 8.5±0.5 in Example 17. The results proved that the magnetic ferroferric oxide nanoparticle (PMAM-ES-MION) in Example 17 had a high r₁ value and a low r₂/r₁ ratio, and could be used as a T₁-weighted MRI contrast agent with good biocompatibility.

### Example 18

### Preparation of Magnetic Ferroferric Oxide Nanoparticle (HA-ES-MION):

20 mL of 2 mg/mL hyaluronic acid HA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as HA-ES-MION.

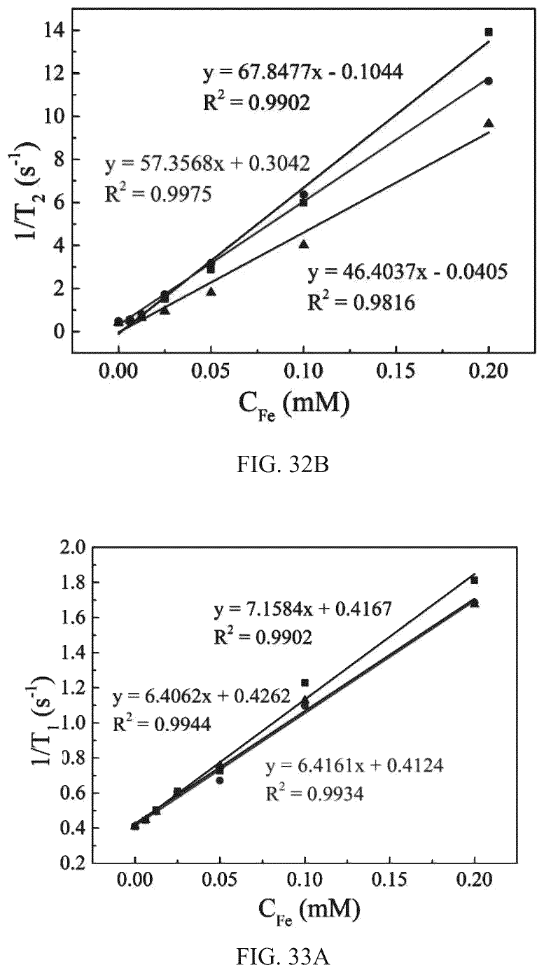

A performance test was carried out on the sample in Example 18. A recovery rate of iron of the sample in Example 18 was calculated to be 89.5%, which indicated that a utilization rate of a raw material was high, so that cost saving was realized. The sample in Example 18 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples HA-ES-MION-1, HA-ES-MION-2 and HA-ES-MION-3 were tested in parallel for three times). As shown in FIG. 31A and FIG. 31B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.5±0.2 mM⁻¹ s⁻¹, an r₂ value was 71.8±5.1 mM⁻¹ s⁻¹, and an r₂/r₁ ratio was 11.0±0.6 in Example 18. The results proved that the magnetic ferroferric oxide nanoparticle (HA-ES-MION) in Example 18 had a high r₁ value and a low r₂/r₁ ratio, and could be used as a T₁-weighted MRI contrast agent with good biocompatibility.

### Example 19

### Preparation of Magnetic Ferroferric Oxide Nanoparticle (SA-ES-MION)

20 mL of 2 mg/mL sodium alginate SA (M_{w}=2,000) aqueous solution was subjected to bubble deoxygenation with nitrogen in a three-necked flask for 1 hour, and then heated at 100° C. to reflux. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as SA-ES-MION.

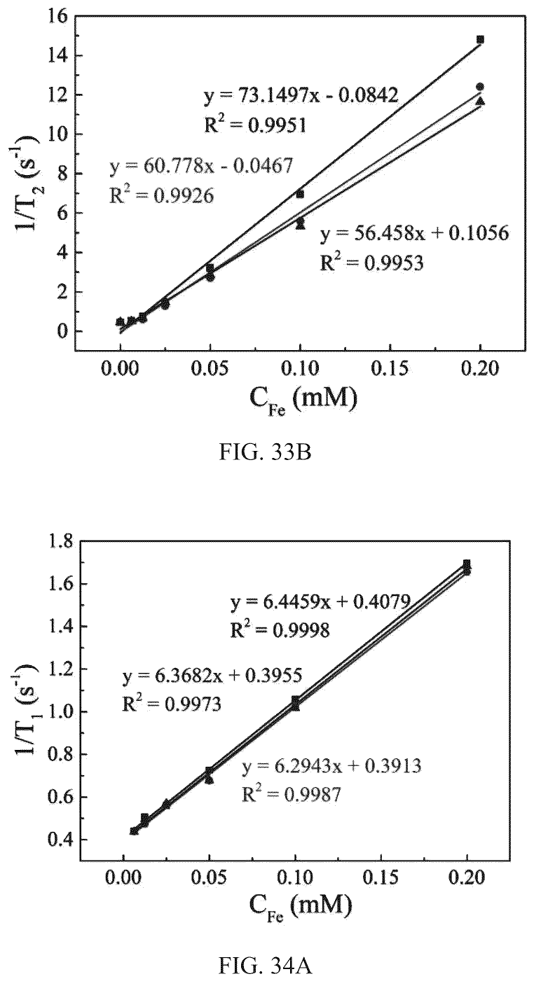

A performance test was carried out on the sample in Example 19. A recovery rate of iron of the sample in Example 19 was calculated to be 89.5%, which indicated that a utilization rate of a raw material was high, so that cost saving was realized. The sample in Example 19 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples SA-ES-MION-1, SA-ES-MION-2 and SA-ES-MION-3 were tested in parallel for three times). As shown in FIG. 32A and FIG. 32B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 7.0±0.4 mM⁻¹ s⁻¹, an r₂ value was 57.2±10.7 mM⁻¹ s⁻¹, and an r₂/r₁ ratio was 8.2±1.7 in Example 19. The results proved that the magnetic ferroferric oxide nanoparticle (SA-ES-MION) in Example 19 had a high r₁ value and a low r₂/r₁ ratio, and could be used as a T₁-weighted MRI contrast agent with good biocompatibility.

### Example 20

### Preparation of Magnetic Ferroferric Oxide Nanoparticle (γ-PGA/PASP-ES-MION):

20 mL of 2 mg/mL polyglutamic acid γ-PGA/polyaspartic acid PASP (polyglutamic acid has a M_{w} of 2,000, and polyaspartic acid has a M_{w} of 7,000 to 8,000) mixture (in a mass ratio of 1:1) solution was heated to reflux in a three-necked flask, and subjected to bubble deoxygenation with nitrogen for 1 hour. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as γ-PGA/PASP-ES-MION.

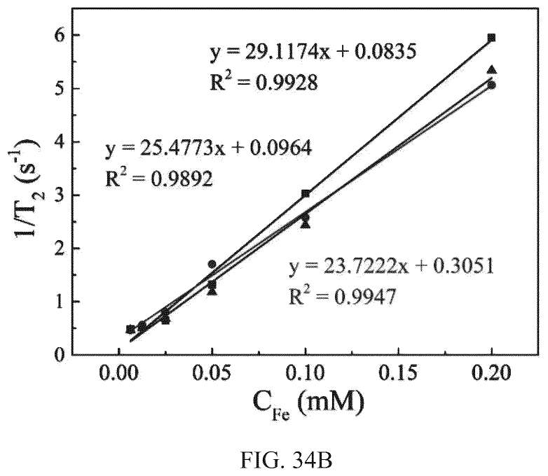

A performance test was carried out on the sample in Example 20. A recovery rate of iron of the sample in Example 20 was calculated to be 93.7%, which indicated that a utilization rate of a raw material was high, so that cost saving was realized. The sample in Example 20 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples γ-PGA/PASP-ES-MION-1, γ-PGA/PASP-ES-MION-2 and γ-PGA/PASP-ES-MION-3 were tested in parallel for three times). As shown in FIG. 33A and FIG. 33B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.7±0.4 mM⁻¹ s⁻¹, an r₂ value was 63.4±8.7 mM⁻¹ s⁻¹, and an r₂/r₁ ratio was 9.5±0.7 in Example 20. The results proved that the magnetic ferroferric oxide nanoparticle (γ-PGA/PASP-ES-MION) in Example 20 had a high r₁ value and a low r₂/r₁ ratio, and could be used as a T₁-weighted MRI contrast agent with good biocompatibility.

### Example 21

### Preparation of Magnetic Ferroferric Oxide Nanoparticle (HPMA/PASP-ES-MION):

20 mL of 2 mg/mL polymaleic acid HPMA/polyaspartic acid PASP (polymaleic acid has a M_{w} of 2,000, and polyaspartic acid has a M_{w} of 7,000 to 8,000) mixture (in a mass ratio of 1:1) solution was heated to reflux in a three-necked flask, and subjected to bubble deoxygenation with nitrogen for 1 hour. 0.4 mL of a mixed aqueous solution containing 0.25 M FeSO₄ and 0.5 M FeCl₃ was added into the flask, and then added with 6 mL of 1% ammonia water. The mixture reacted for 1 hour under magnetic stirring at 100° C., and was allowed to stand and cooled. The sample after the reaction was dialyzed and purified, and the final sample was marked as HPMA/PASP-ES-MION.

A performance test was carried out on the sample in Example 21. A recovery rate of iron of the sample in Example 21 was calculated to be 90.1%, which indicated that a utilization rate of a raw material was high, so that cost saving was realized. The sample in Example 21 was prepared into 6 aqueous solutions with different concentrations, and in-vitro imaging tests were carried out by a clinical 3.0 T MRI system (Philips, Ingenia) to obtain longitudinal relaxation time T₁ and transverse relaxation time T₂ (three samples HPMA/PASP-ES-MION-1, HPMA/PASP-ES-MION-2 and HPMA/PASP-ES-MION-3 were tested in parallel for three times). As shown in FIG. 34A and FIG. 34B, a change relationship between 1/Tᵢ and a concentration of iron was drawn, and a slope of a fitted line was a magnetic relaxation rate of Tᵢ (i=1 or 2). Thus, it could be seen that, under the 3.0 T MRI system, an r₁ value was 6.4±0.1 mM⁻¹ s⁻¹, an r₂ value was 26.1±2.8 mM⁻¹ s⁻¹, and an r₂/r₁ ratio was 4.1±0.4 in Example 21. The results proved that the magnetic ferroferric oxide nanoparticle (HPMA/PASP-ES-MION) in Example 21 had a high r₁ value and a low r₂/r₁ ratio, and could be used as a T₁-weighted MRI contrast agent with good biocompatibility.

The above examples are the preferred implementations of the present disclosure, but the implementations of the present disclosure are not limited by the above examples. Any other changes, modifications, substitutions, combinations, and simplifications made without departing from the spirit and principle of the present disclosure should be equivalent substitute modes, and should be included in the scope of protection of the present disclosure.

1. A magnetic ferroferric oxide nanoparticle, comprising ferroferric oxide and a hydrophilic macromolecule, wherein the ferroferric oxide and the hydrophilic macromolecule are in at least one of the following relationships (1) and (2): (1) the hydrophilic macromolecule is adsorbed on a surface of the ferroferric oxide; and (2) the ferroferric oxide and the hydrophilic macromolecule are mutually embedded or occluded; and
the magnetic ferroferric oxide nanoparticle simultaneously has the following properties 1) to 4):
1) an average particle size is greater than 2 nm and less than 5 nm;
2) an electrokinetic potential is less than or equal to −10 mV;
3) a hydrodynamic diameter is less than or equal to 20 nm; and
4) a longitudinal magnetic relaxation rate r₁ value under a magnetic field intensity of 3.0 T is greater than 5 mM⁻¹ s⁻¹; and a longitudinal magnetic relaxation rate r₁ value under a magnetic field intensity of 1.0 T is greater than 10 mM⁻¹ s⁻¹.

2. The magnetic ferroferric oxide nanoparticle according to claim 1, wherein the hydrophilic macromolecule comprises any one or a copolymer or mixture of several of a carboxylic acid-containing macromolecule, an amino-containing macromolecule, a hydroxyl-containing macromolecule, an amide-containing macromolecule and a polysaccharide.

3. The magnetic ferroferric oxide nanoparticle according to claim 2, wherein the carboxylic acid-containing macromolecule comprises any one or more of polyglutamic acid, polyaspartic acid, polymaleic acid, poly(2-ethylacrylic acid) and polyepoxysuccinic acid.

4. The magnetic ferroferric oxide nanoparticle according to claim 2, wherein the amino-containing macromolecule comprises any one or more of polylysine, polyhistidine, poly-L-arginine and polydimethyl diallyl ammonium chloride.

5. The magnetic ferroferric oxide nanoparticle according to claim 2, wherein the hydroxyl-containing macromolecule comprises any one or more of polyserine, polythreonine, polytyrosine and tannic acid.

6. The magnetic ferroferric oxide nanoparticle according to claim 2, wherein the amide-containing macromolecule comprises any one or more of polyglutamine, polyasparamide, polyacrylamide and polymethacrylamide.

7. The magnetic ferroferric oxide nanoparticle according to claim 2, wherein the polysaccharide comprises one or two of hyaluronic acid and sodium alginate.

8. A preparation method for the magnetic ferroferric oxide nanoparticle according to claim 1, comprising a step of: coprecipitating ferrous ions and ferric ions to form the magnetic ferroferric oxide nanoparticle by using the hydrophilic macromolecule as a stabilizer.

9. The preparation method according to claim 8, wherein the preparation method for the magnetic ferroferric oxide nanoparticle comprises the following steps of: heating a hydrophilic macromolecule solution, then mixing the hydrophilic macromolecule solution after being subjected to heating with an iron ion mixed solution comprising ferrous ions and ferric ions for a coordination reaction, and then adding an alkali liquor for a coprecipitation reaction to obtain the magnetic ferroferric oxide nanoparticle.

10. A magnetic resonance imaging contrast agent comprising the magnetic ferroferric oxide nanoparticle according to claim 1.

11. The magnetic ferroferric oxide nanoparticle according to claim 1, wherein a Zeta potential of the magnetic ferroferric oxide nanoparticle is less than or equal to −30 mV.

12. The magnetic ferroferric oxide nanoparticle according to claim 1, wherein a hydrodynamic diameter of the magnetic ferroferric oxide nanoparticle is less than or equal to 20 nm.

13. The magnetic ferroferric oxide nanoparticle according to claim 1, wherein a molecular weight of the hydrophilic macromolecules is 1,000 to 10,000.

14. The preparation method according to claim 9, wherein in the iron ion mixed solution, a concentration of ferrous ions is 30 mM to 500 mM, and a concentration of ferric ions is 60 mM to 1,000 mM.

15. The preparation method according to claim 9, wherein a concentration of the hydrophilic macromolecule solution is 0.1 mg/mL to 20 mg/mL.

16. The preparation method according to claim 9, wherein the ferrous ions are obtained by hydrolysis of water-soluble ferrous salt, and the water-soluble ferrous salt comprises any one or more of ferrous chloride, ferrous nitrate, ferrous bromide and ferrous sulfate.

17. The preparation method according to claim 9, wherein the ferric ions are obtained by hydrolysis of water-soluble ferric salt, and the water-soluble ferric salt comprises any one or more of ferric chloride, ferric nitrate, ferric bromide and ferric sulfate.

18. The preparation method according to claim 9, wherein after the alkali liquor is added into a mixed solution of the iron ion mixed solution and the hydrophilic macromolecule solution, a pH value of a reaction system is 8 to 10.

19. The preparation method according to claim 9, wherein the alkali liquor comprises at least one of sodium hydroxide and an aqueous solution thereof, potassium hydroxide and an aqueous solution thereof, and ammonia water.

20. The preparation method according to claim 9, wherein the hydrophilic macromolecule solution is heated at a temperature of 25° C. to 100° C., the coprecipitation reaction is conducted at a temperature of 25° C. to 100° C.
